(19) United States
(12) Patent Application Publication
(10) Pub. No.: US 2010/0022584 A1
(43) Pub. Date: Jan. 28, 2010

(54) Modulation of Glutamine Synthetase Activity

(76) Inventors: Colin Peter Kenyon, Randburg (ZA); Lyndon Carey Oldfield, Kempton Park (ZA)

Correspondence Address: LADAS & PARRY LLP, 26 WEST 61ST STREET, NEW YORK, NY, 10023, US

(21) Appl. No.: 12/224,862

(22) PCT Filed: Mar. 15, 2006
(86) PCT No.: PCT/IB2006/000561

§ 371 Date: Apr. 13, 2009

(51) Int. Cl. A61K 31/47; G06G 7/48; C12Q 1/34; C40B 30/00; C12N 9/99; C12N 1/20; A61K 31/195; A61K 31/40; A61K 31/415

(52) U.S. Cl. 514/311; 703/11; 435/18; 506/7; 435/184; 435/252.1; 514/564; 514/412; 514/396

(57) ABSTRACT

Methods of screening and designing compounds as inhibitors of glutamine synthetase are provided herein. Compounds, e.g., serine protease inhibitors, and compositions comprising the same, that are useful for the treatment, prevention, and/or amelioration of bacterial infections, including Mycobacterium tuberculosis, are also provided.

### TECHNICAL FIELD

This invention relates to materials and methods for inhibiting glutamine synthetase activity, and more particularly to materials and methods for inhibiting the second catalyzed reaction of glutamine synthetase, the formation of glutamine, through a postulated mechanism involving the nucleophilic attack of ammonia on a γ-glutamyl enzyme acyl intermediate, and to materials and methods for designing and using such inhibitors.

### BACKGROUND

The enzyme glutamine synthetase (GS, EC 6.3.1.2) is a central enzyme involved in nitrogen metabolism and catalyses the reversible conversion of L-glutamate, ATP and ammonia into L-glutamine, ADP, and inorganic phosphate. The reaction is mediated via a γ-glutamyl phosphate intermediate. The enzyme requires at least two divalent metal ions (Me2+) per subunit; the binding sites of the divalent metal ions are referred to as n1 and n2 herein. GS catalyses the biosynthesis of glutamine from glutamate as follows:

$$Glutamate + {NH}_{4}^{+} + ATP {\to }^{{Me}^{2 +}} Glutamine + ADP + {P}_{i} + {H}_{2} O ,$$

where Me²⁺ represents a divalent metal cation selected from magnesium or manganese. This reaction is often referred to as the ‘biosynthetic’ or ‘forward’ reaction, and is considered the most physiologically relevant reaction that glutamine synthetase catalyses.

Formation of the γ-glutamyl phosphate occurs by the transfer of the γ-phosphate of ATP to the γ-carboxylate group of glutamate. Efficient phosphoryl transfer between these two negatively charged groups is co-ordinated by the n2 metal. The currently accepted mechanism posits that this is followed by phosphate displacement by ammonia to give inorganic phosphate and glutamine:

ATP+Glutamate→ADP+γ-Glutamate-P

ADP+γ-Glutamate-P+NH₄→ADP+Glutamine+Pᵢ.

Structurally, the GS enzyme from Escherichia coli is a large metalloenzyme (˜600,000 Mr) with 12 identical subunits arranged in two face-to-face hexagonal rings, with two active sites formed between two monomers, for a total of twelve active sites. Each active site can be described as a “bifunnel” in which ATP and glutamate bind at opposite ends. The ATP binding site is referred to as the top of the bifunnel, because it opens to the external six-fold surface of the glutamine synthetase molecule. The two divalent cation binding sites, n1 and n2, are found at the joint of the bifunnel. The n2 ion is involved in phosphoryl transfer, while the n1 ion stabilises glutamine synthetase in its active form and plays a role in the binding of glutamate. The affinity for metal ions at the n1 site is 50 times greater than at the n2 site, caused by the greater negative charge toward the bottom half of the bifunnel in the vicinity of n1. The n1 metal ion has three glutamate residue side chains—131, 212 and 220—as ligands, while the n2 metal ion has two glutamate residue ligands, 129 and 357, as well as histidine 269 (Abell, L M, J Schineller, P J Keck and J Villafranca (1995) Biochemistry 34: 16695-16702). The amino acids that serve as metal ion ligands are highly conserved in glutamine synthetases from various sources (Pesole G, M P Bozzetti, C Lanave, G Preparata and C Saccone (1991) Proceedings of the National Academy of Sciences, USA 88: 522-526). (Unless specifically indicated otherwise, all GS amino acid residue numbers identified herein refer to the residues of E. coli GS. Given the homology among bacterial GS polypeptide sequences, one having skill in the art could determine the corresponding residues of interest of GS in other species by using methods such as homology alignments or molecular modeling.)

Conformational changes and side-chain movements have been described for glutamine synthetase crystals soaked in solutions with various ligands (Liaw S-H, J J Villafranca and D Eisenberg (1993b) Biochemistry 33:7999-8003; Liaw S-H and D Eisenberg (1994) Biochemistry 33: 675-681; Liaw S-H, G Jun and D Eisenberg (1994) Biochemistry 33: 11184-11188). These residues are absolutely conserved among glutamine synthetase in both lower and higher organisms, and appear to play key roles in the mechanism of the biosynthetic reaction (Eisenberg D, H S Gill, G M U Pfluegl and S H Rotstein (2000) Biochemica et Biophysica Acta 1477: 122-145).

The structure of the glutamine synthetase dodecamer, as described by Eisenberg (2000) is believed to contain several loops of functional importance (Eisenberg D, H S Gill, G M U Pfluegl and S H Rotstein (2000) Biochemica et Biophysica Acta 1477: 122-145). These loops, with residue numbers corresponding to the E. coli residues, are as follows:
A loop consisting of the hydrophilic residues 156-173, which protrudes into the central channel of the dodecamer and is a site for proteolysis and ADP-ribosylation.
A second loop, known as the adenylylation loop, contains tyrosine 397, which is covalently modified by the addition of AMP. This loop lies just outside the bottom entrance to the bifunnel.
The glutamate 327 flap, consisting of residues 323 to 330, which guards the glutamate entrance to the active site. This ‘flap’ closes the active site, shielding the γ-glutamyl phosphate intermediate from hydrolysis. When the flap is closed, the glutamate 327 carboxylate forms part of the ammonium site. Aspartate 50′ deprotonates the ammonium ion, forming ammonia. Ammonia then attacks the γ-glutamyl phosphate intermediate thereby forming a tetrahedral intermediate at the transition state. The glutamate 327 flap accepts a proton from the 6-amino group of the tetrahedral intermediate, yielding glutamine.
A loop containing aspartate 50′ is located on the N-terminal domain (residues 1-100). Each active site is formed at the interface between the C-terminal domain of one subunit and the N-terminal domain of an adjacent subunit within a dodecameric ring, resulting in most of the active site being formed by residues of the C-terminal domain. Aspartate 50′ is in the N-terminal portion of the active site, and is believed to bind the ammonium substrate and then to accept a proton from ammonium, resulting in the formation of ammonia which can then attack the phosphorylated-glutamyl intermediate. The position of aspartate 50′ is controlled by nucleotide binding. Both ADP and ATP enter the active site from the top of the bifunnel, with the phosphate chain pointing into the bifunnel. ADP induces arginine 359 interaction with aspartate 50′ and stimulates arginine 344 interaction with aspartate 64′, providing additional contacts for inter-subunit stabilisation within a ring. The movement of aspartate 50′ helps in the formation of the ammonium binding site, and the movement of arginine 339 possibly assists phosphoryl transfer and phosphate binding. It is thought that ADP binding also increases the affinity for glutamate by inducing the movement of arginine 359 toward one of the γ-carboxylate oxygens of glutamate. Finally, the β-phosphate of ADP shifts glutamate 129 toward the n2 ion, histidine 269 and histidine 271.
Studies in which the metal ion ligand, histidine 269, was replaced by other amino acid residues, namely aspartate, asparagine, glutamate and glutamine, by site-directed mutagenesis have been carried out. All of the mutant enzymes showed little conformational change, and were still capable of binding two metal ions. The replacement of histidine 269 with neutral ligands such as asparagine and glutamine slightly altered the dissociation constants (3- to 4-fold), while substitution with glutamate decreased the dissociation constant slightly. The mutations had little effect on the substrate Kₘ's except in the case of H269E, whose kₘ exhibited a 1000-fold increase over that of the wild type (Abell, L M, J Schineller, P J Keck and J Villafranca (1995) Biochemistry 34: 16695-16702).

Asparagine 264 is found on a flexible loop (residues 255-266) near the glutamate entrance at the lower end of the bifunnel and is adjacent to the glutamate 327 flap. When glutamate binds, the side chain swings away toward the ε-amino group of lysine 176, and was found to also be true when alanine, glycine and glutamine complex with glutamine synthetase.

As a result of the elucidation of these loops described above, it is possible to describe the enzymatic reaction mechanism of glutamine synthetase as a series of loop and side-chain movements. In the proposed reaction mechanism, ATP binds at the top of the bifunnel, so that its terminal phosphate group binds adjacent to the n2 ion. This binding of ATP results in the movement of the aspartate 50′ loop toward the site to which an ammonium ion will subsequently bind. Arginine 359 then moves toward the site to which the γ-carboxylate group of glutamate will subsequently bind. Both these movements increase the affinity for glutamate and ammonium binding. Following this, glutamate enters from the bottom of the bifunnel and binds above the glutamate 327 flap, with its γ-carboxylate group binding adjacent to the n1 ion. The amino group of glutamate shifts the asparagine 264 loop, aiding serine 52′ on the aspartate 50′ loop, to stabilise the flap. The active site is now closed and is shielded from water, and ammonium binding is complete. The γ-phosphate of ATP is transferred to the γ-carboxylate of glutamate, thereby forming the intermediate. The two positive charged metal ions and arginine 359 participate in phosphoryl transfer by polarising the γ-phosphate group of ATP making the γ-phosphorous more positive. An ammonium ion enters the bifunnel and binds in the negatively charged pocket created by glutamate 327, aspartate 50′, tyrosine 19, glutamate 212 and serine 53′. The side chain of aspartate 50′ deprotonates the ammonium ion, forming ammonia, which then attacks the carbon of the γ-glutamyl phosphate intermediate, which results in the release of a phosphate group. A salt-bridge is now formed between the tetrahedral adduct and glutamate 327, which then accepts a proton from the adduct, thereby neutralising the salt-bridge and forming glutamine. Finally, the glutamate 327 flap opens and glutamine is released.

Three distinct forms of glutamine synthetase occur: GSI, GSII, and GSIII. The GSI form is found only in bacteria (eubacteria) and archaea (archaebacteria). GSII occurs in eukaryotes and certain soil-dwelling bacteria, while GSIII genes have been found only in a few bacterial species. There are two significant GSI sub-divisions: GSI-α and GSI-β. The GSI-A genes are found in thermophilic bacteria, low G+C gram-positive bacteria, and Euryarchaeota (including methanogens, halophiles and some thermophiles), while the GSI-β genes are found in all other bacteria. The GSI-β enzyme is regulated via an adenylylation/deadenylylation cascade, and also contains a 25 amino acid insertion sequence that does not occur in the GSI-α form. Bacteria that have a GSI-β gene include Corynebacterium diphtheriae, Neisseria gonorrhoeae, Escherichia coli, Salmonella typhinurium, Salmonella typhi, Klebsiella pneumoniae, Serratia marcescens, Proteus vulgaris, Shigella dysenteriae, Vibrio cholerae, Pseudomonas aeruginosa, Alcaligenes faecalis, Helicobacter pylori, Haemophilus influenzae, Bordetella pertussis, Bordella bronchiseptia, Neisseria meningitides, Brucella melitensis, Mycobacterium tuberculosis, Mycobacterium leprae, Treponema pallidum, Leptospira interrogans, Acetinomyces israelii, Nocardia esteroides, Thiobacillus ferrooxidans, Azospirillum brazilensis, Anabaena sp., Fremyella diplosiphon, and Streptomyces coelicolor. Examples of organisms in the GSI-α sub-division include Bacillus cereus, Bacillus subtilis, Bacillus anthracis, Streptococcus pneumoniae, Streptococcus pyogenes, Staphylococcus aereus, Clostridium botulinum, Clostridium tetani and Clostridium perfringens.

In E. coli and other bacteria that have the GSI-β gene, GS activity is regulated by adenylylation of between 1 and 12 GS subunits. The site of adenylylation (equivalent to Tyr397 in E. coli) appears to be highly conserved across all prokaryotic bacteria, while the extent of adenylylation is a function of the availability of nitrogen and carbon energy source in the culture media.

Glutamine synthetase with 10 to 12 adenylylated subunits occurs in cells grown in the presence of an excess of nitrogen and carbon limitation. The adenylylation of GS also changes the enzyme's specificity for divalent metal ion from Mg²⁺ to Mn²⁺.

The development and therapeutic use of serine protease inhibitors has occurred in diseases including hepatitis C virus (HCV) and diseases related to disorders of the blood coagulation cascade, such as myocardial infarction, stroke and deep vein thrombosis. Inhibitors of thrombin, factor Xa and the factor VIIIa complex have been developed for use as orally bio-available anticoagulants in thromboembolic disorders and in the prevention of venous and arterial thrombosis. These enzymes have structurally similar active sites, as they all belong to the trypsin family of serine proteases.

A number of classes of inhibitors have been designed specifically to target these serine protease enzymes. Included in these are the peptidyl-arginine aldehyde derivatives (Bajusz, S., et al (1997) WO 97/46576), pyrrolopyrrrolidene derivatives (Cooke, S. H. et al (1998), WO 99/12935), quinolinones (Dudley, D. A. and J. J. Edmunds (1999), WO 99//50263) and Pastor R. M., Artis, D. R. and A. G. Olivera (2002), WO 02/22575). Dual inhibitors to thrombin and factor Xa include compounds containing 1-methyl benzimidazole moieties using 4-(1-methyl-benzimidazole-z-yl)-methylamino-benzoamidine as the basic scaffold (Nar, H., et al. (2001) Structure, 9: 29-37).

### SUMMARY

This disclosure is based, in part, on the finding that glutamine synthetase employs one of two serine protease-like catalytic triads, depending on its adenylylation state, to catalyze the reaction of ammonia with the γ-glutamyl phosphate formed in the first enzymatic step. As used herein, the active site in GS where glutamine is formed is referred to herein as the glutamine formation site. While not being bound by any theory, the inventors believe that a mechanism by which the adenylylated or deadenylylated state of the enzyme affects the enzymatic specificity for either MgATP/NH₄⁺ or Mn₂ATP/NH₃ is by inducing a switch between the two putative catalytic triads. Depending on the adenylylation state of the enzyme, one of the two triads is involved in a nucleophilic attack by an activated serine on the carboxylic-phosphoric acid anhydride intermediate of the first reaction, the γ-glutamyl phosphate, to form an acyl enzyme intermediate. The glutamyl acyl intermediate then undergoes nucleophilic attack by NH₃, releasing the glutamine from the surface of the enzyme.

The presence of two catalytic triads is consistent with the fact that the glutamine synthetase from E. coli has two affinity constants for ammonia (Meek T D and J J Villafranca (1980) Biochemistry 19: 5513-5519). The solution chemistry of the NH₄⁺/NH₃ also affects the regulation of GS, with the adenylylated enzyme being produced under conditions of nitrogen excess and carbon limitation, and the deadenylylated enzyme under conditions of nitrogen limitation and carbon excess. At low ammonium salt concentrations, the NH₄⁺ dissociates to NH₃+H⁺. The NH₃ is a strong nucleophile and capable of carrying out the nucleophilic attack on the proposed γ-glutamyl acyl enzyme intermediate. Moreover, site-directed mutagenesis experiments and inhibition of GS activity by the known serine protease inhibitors AEBSF and PMSF, as shown below, further demonstrated that the final step of the glutamine synthetase reaction can be mediated by the presence of the two serine protease-like catalytic triads.

Knowledge of serine protease catalytic triad chemistry and reaction mechanisms can thus be coupled with knowledge of the GS structure, including the GS glutamine formation active site structure, in the design of inhibitors specific to adenylylated and/or deadenylylated glutamine synthetase. Accordingly, computer-assisted methods to design test inhibitor compounds are provided herein, as well as methods for in vitro and in vivo screening of the inhibitory activity of test compounds. Compounds so designed or screened can be used to inhibit adenylylated or deadenylylated GS activity and consequently to treat, prevent, or ameliorate bacterial infections in mammals. As the bacterial GSI-β enzymes are regulated via an adenylylation/deadenylylation cascade, but the mammalian GSII enzymes are not, compounds having an inhibitory effect on adenylylated GS, but having no or only a minimal inhibitory effect on deadenylylated GS, can also be used to selectively inhibit GSI-β bacterial cell growth while minimally negatively impacting mammalian cells. Compounds and compositions for inhibiting GS activity, including adenylylated and deadenylylated GS activity; for inhibiting or preventing bacterial growth in vitro and in vivo; and for treating, preventing, or ameliorating bacterial infections in mammals are also provided herein.

Accordingly, in one embodiment, a computer-assisted method of generating a test inhibitor of the glutamine formation active site activity of a glutamine synthetase polypeptide is provided. The method uses a programmed computer comprising a processor and an input device, and includes:

(a) inputting on the input device data comprising a structure of a glutamine formation active site;

(b) docking into the glutamine formation active site a test inhibitor molecule using the processor; and

(c) determining, based on the docking, whether the test inhibitor molecule would inhibit the glutamine formation active site activity. The method can further comprise docking into the active site a γ-glutamyl phosphate moiety, and/or producing a test inhibitor determined by step (c) to inhibit the glutamine formation active site activity and evaluating the inhibitory activity of the test inhibitor on a glutamine synthetase polypeptide in vitro. In vitro evaluation comprises use of an assay capable of measuring ATP hydrolysis, ADP formation, AMP formation, glutamate utilization, or glutamine formation. In some embodiments, the method can further include evaluating the differential inhibitory activity of the test inhibitor on an adenylylated glutamine synthetase polypeptide relative to a deadenylylated glutamine synthetase polypeptide in vitro, and/or producing the test inhibitor and evaluating the inhibitory activity of the test inhibitor on the growth of a bacterium comprising a GSI-α or a GSI-β glutamine synthetase gene. A GSI-β bacterium can be selected from the group consisting of Corynebacterium diphtheriae, Neisseria gonorrhoeae, Escherichia coli, Salmonella typhinurium, Salmonella typhi, Klebsiella pneumoniae, Serratia marcescens, Proteus vulgaris, Shigella dysenteriae, Vibrio cholerae, Pseudomonas aeruginosa, Alcaligenes faecalis, Helicobacter pylori, Haemophilus influenzae, Bordetella pertussis, Bordella bronchiseptia, Neisseria meningitides, Brucella melitensis, Mycobacterium tuberculosis, Mycobacterium leprae, Treponema pallidum, Leptospira interrogans, Acetinomyces israelii, Nocardia esteroides, Thiobacillus ferrooxidans, Azospirillum brazilensis, Anabaena sp., Fremyella diplosiphon, and Streptomyces coelicolor, and a GSI-α bacterium can be selected from the group consisting of Bacillus cereus, Bacillus subtilis, Bacillus anthracis, Streptococcus pneumoniae, Streptococcus pyogenes, Staphylococcus aereus, Clostridium botulinum, Clostridium tetani and Clostridium perfringens.

In some embodiments, the method can include evaluating the inhibitory activity of the test inhibitor on the growth of a eukaryotic cell, such as a mammalian (e.g., human) cell.

In another aspect, provided herein is a method of generating a compound that inhibits the glutamine formation active site activity of a glutamine synthetase polypeptide, the method comprising:

(a) providing a three-dimensional structure of a glutamine formation active site of a glutamine synthetase polypeptide; and

(b) designing, based on the three-dimensional structure, a test compound capable of inhibiting the interaction between the glutamine formation active site and a γ-glutamyl phosphate intermediate. In some embodiments, the test compound is capable of inhibiting the interaction between an adenylylated catalytic triad site of the glutamine formation active site and a γ-glutamyl phosphate intermediate, or of inhibiting the interaction between an deadenylylated catalytic triad site of the glutamine formation active site and a γ-glutamyl phosphate intermediate. In some embodiments, the test compound is capable of inhibiting the formation of an acyl-enzyme intermediate in the adenylylated catalytic triad site of the glutamine formation active site, or of inhibiting the formation of the acyl-enzyme intermediate in the deadenylylated catalytic triad site of the glutamine formation active site. The method can further include producing the test compound of step (b) and evaluating the inhibitory activity of the test compound on a glutamine synthetase polypeptide in vitro, and/or evaluating the inhibitory activity of the test compound on the growth of a bacterium comprising a GSI-α or a GSI-β glutamine synthetase gene. In some cases, the method can include evaluating the inhibitory activity of the test compound on the growth of a eukaryotic cell, such as a mammalian (e.g., human) cell.

In another aspect, provided herein is a method of generating a test compound that inhibits a catalytic triad site activity of a glutamine formation active site of a glutamine synthetase polypeptide. The method can include (a) providing a three-dimensional structure comprising a catalytic triad site of a glutamine formation active site; and (b) designing, based on the three-dimensional structure, a test compound capable of forming an acyl-enzyme intermediate with a residue of the catalytic triad site. In some embodiments, the test compound is capable of forming an acyl-enzyme intermediate with a residue in the structure corresponding to Ser52 or Ser53 of the glutamine synthetase polypeptide of E. coli.

In another embodiment, a method of screening a test protease inhibitor compound in vitro to determine whether or not it inhibits the glutamine formation active site activity of a glutamine synthetase polypeptide is provided, where the method includes:

(a) contacting a glutamine synthetase polypeptide with a test protease inhibitor compound; and

(b) determining whether or not the glutamine formation active site activity of the glutamine synthetase polypeptide is reduced relative to the activity of a glutamine synthetase polypeptide that has not been contacted with the test serine protease inhibitor compound. The glutamine formation site activity can be measured by using an assay capable of measuring ATP hydrolysis, ADP formation, AMP formation, glutamate utilization, or glutamine formation. The test protease inhibitor compound can be a test serine protease inhibitor compound. In some embodiments, a library of test protease inhibitors (e.g., a library of test serine protease inhibitors) are contacted, individually, with the glutamine synthetase polypeptide.

In another embodiment, an in vitro method for inhibiting the glutamine formation active site activity of a GS polypeptide is provided, where the method includes contacting a GS polypeptide with a composition comprising a serine protease inhibitor. The serine protease inhibitor can be a peptidyl-arginine aldehyde derivative, a pyrrolopyrrrolidene derivative, a quinolinone derivative, or a 1-methyl benzimidazole derivative.

An in vitro method for inhibiting growth of a bacterium comprising a GSI-α or a GSI-β gene is also provided, where the method includes contacting the bacterium with a composition comprising a serine protease inhibitor.

In yet another embodiment, a method for treating, preventing, or ameliorating one or more symptoms or disorders associated with a bacterial infection in a mammal is provided, where the bacterial infection is from a bacterium comprising a GSI-α or a GSI-β gene. The method can include administering to the mammal a composition comprising a serine protease inhibitor.

Moreover, an in vivo method for inhibiting the glutamine formation site activity of a glutamine synthetase polypeptide is also provided, where the method includes administering a composition comprising a serine protease inhibitor to a mammal that is suspected of suffering or is suffering from a bacterial infection, where the bacterial infection is from a bacterium comprising a GSI-α or GSI-β gene.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can also be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention, e.g., inhibiting adenylylated GS activity and thus methods for treatment of bacterial infections, will be apparent from the following description, from the drawings and from the claims.

### DESCRIPTION OF DRAWINGS

FIG. 1 shows a postulated reaction mechanism of adenylylated glutamine synthetase at the glutamine formation active site at high concentrations of ammonia, requiring the deprotonation of the NH₄⁺. The mechanism starts at the upper left with a non-covalent Michaelis complex. Subsequently, formation of the first tetrahedral transition state occurs, followed by formation of the acyl-enzyme intermediate, release of phosphate, and deprotonation on ammonium by Asp50′ or by the acyl intermediate. Nucleophilic attack of the acyl intermediate by ammonia and the formation of the second tetrahedral transition state then occurs, followed by release of the glutamine and return to non-covalent Michaelis complex.

FIG. 2 shows a postulated reaction mechanism of deadenylylated glutamine synthetase at the glutamine formation active site at low concentrations of ammonia. The mechanism starts at the upper left with a non-covalent Michaelis complex. Subsequently, formation of the first tetrahedral transition state occurs, followed by formation of the acyl-enzyme intermediate and the release of phosphate. Nucleophilic attack of the carbonium ion by ammonia and the formation of the second tetrahedral transition state then occurs, followed by release of the glutamine and return to non-covalent Michaelis complex.

### DETAILED DESCRIPTION

### Definitions

The terms “GS polypeptide” and “glutamine synthetase polypeptide” are used interchangeably herein, and unless otherwise indicated, refer to a bacterial GSI polypeptide, e.g., from a GSI-α or GSI-β bacterium. Unless otherwise indicated, the term encompasses the full length polypeptide and fragments thereof. In addition, as will be recognized by those having skill in the art, GS functions as a dodecamer in vivo and GS active sites can be made up of amino acids from more than one monomer. Accordingly, the term also encompasses multimers (e.g., dimers, trimers, tetramers, pentamers, hexamers, heptamers, octamers, nonamers, decamers, undecamers, and dodecamers) of full length GS, multimers of fragments of GS, one or more residues that are part of one or more of the active sites of GS (e.g., a collection of residues that may not be contiguous in the primary sequence of GS and/or that are from distinct monomers, but that make up at least a part of an active site of GS), and multimers of such collections.

“Polypeptide” and “protein” are used interchangeably and mean any peptide-linked chain of amino acids, regardless of length or post-translational modification.

The term “isolated” can refer to a polypeptide which either has no naturally-occurring counterpart or has been separated or purified from components which can naturally accompany it, e.g., in tissues such as pancreas, liver, spleen, ovary, testis, muscle, joint tissue, neural tissue, gastrointestinal tissue or tumor tissue (e.g., breast cancer or colon cancer tissue); or body fluids such as blood, serum, or urine, or from bacterial or fungal culture. Typically, a polypeptide is considered “isolated” when it is at least 70%, by dry weight, free from the proteins and other naturally-occurring organic molecules with which it is naturally associated.

Preferably, a preparation of a polypeptide is at least 80%, more preferably at least 90%, and most preferably at least 99%, by dry weight, the polypeptide. Since a polypeptide that is chemically synthesized is, by its nature, separated from the components that naturally accompany it, a synthetic polypeptide is “isolated.”

An isolated polypeptide can be obtained, for example, by extraction from a natural source (e.g., from tissues); by expression of a recombinant nucleic acid encoding the polypeptide; or by chemical synthesis. A polypeptide that is produced in a cellular system different from the source from which it naturally originates is “isolated,” because it will necessarily be free of components which naturally accompany it. The degree of isolation or purity can be measured by any appropriate method, e.g., column chromatography, polyacrylamide gel electrophoresis, or HPLC analysis.

Prior to testing, any polypeptide can undergo modification, e.g., adenylylation, phosphorylation or glycosylation, by methods known in the art and as described herein.

As used herein, “pharmaceutically acceptable derivatives” of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs.

Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, nitrates, borates, methanesulfonates, benzenesulfonates, toluenesulfonates, salts of mineral acids, such as but not limited to hydrochlorides, hydrobromides, hydroiodides and sulfates; and salts of organic acids, such as but not limited to acetates, trifluoroacetates, maleates, oxalates, lactates, malates, tartrates, citrates, benzoates, salicylates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a bacterial infection, e.g., Mycobacterium tuberculosis infection, are ameliorated or otherwise beneficially altered. Treatment also encompasses any pharmaceutical use of the compositions herein, such as uses for treating diseases, disorders, or ailments in which a bacterial infection is implicated.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, IC₅₀ refers to an amount, concentration or dosage of a particular test compound that achieves a 50% inhibition of a maximal response in an assay that measures such response.

As used herein, the term Kᵢ represents the dissociation constant of an enzyme/inhibitor complex. It is theoretically independent of the substrate against which the inhibitor is tested. Kᵢ can be calculated from an IC₅₀ using the equation: Kᵢ=IC₅₀*Kₘ/(S+Kₘ), where S is the concentration of substrate, and Kₘ is the substrate concentration (in the absence of inhibitor) at which the velocity of the reaction is half-maximal. The Kᵢ of an inhibitor for inhibition of a particular substrate (fixed Kₘ) is constant. As used herein, EC₅₀ refers to a drug concentration that produces 50% of inhibition, and CC₅₀ refers to a drug concentration that produces 50% of toxicity.

As used herein, a prodrug is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

As used herein, “substantially pure” with respect to a compound means sufficiently homogeneous to appear free of readily detectable impurities as determined by standard methods of analysis, such as thin layer chromatography (TLC), gel electrophoresis, high performance liquid chromatography (HPLC) and/or mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

### Methods of Designing Inhibitors to the GSA Active Site

Provided herein are methods, including computer-based methods, for designing compounds that bind to and/or inhibit the glutamine formation active site of GS, particularly an GS polypeptide from a GSI bacterium. As described further herein, the glutamine formation active site of GS is proposed to catalyze a nucleophilic attack by ammonia on a γ-glutamyl acyl enzyme intermediate to form glutamine. Such a glutamine formation active site can include one or more serine-like catalytic triads, such as the adenylylated and/or deadenylylated GS catalytic triads described herein. The inventors have postulated that glutamine synthetase employs one of two serine protease-like catalytic triads, depending on the adenylylation state, to catalyze the reaction of ammonia with the γ-glutamyl phosphate formed in the first GS enzymatic step. Depending on the adenylylation state of the enzyme, one of the two triads is involved in a nucleophilic attack by an activated serine on the carboxylic-phosphoric acid anhydride intermediate of the first reaction, the γ-glutamyl phosphate, to form an acyl enzyme intermediate. The glutamyl acyl intermediate then undergoes nucleophilic attack by NH₃, releasing the glutamine from the surface of the enzyme.

Accordingly, by analyzing one or both of the catalytic triads, one of skill in the art would know how to use standard molecular modeling or other techniques to identify peptides, peptidomimetics, and small-molecules that would bind to and/or inhibit the glutamine formation active site of GS, such as by binding to and/or inhibiting the activity of one or both of the catalytic triads contained therein, to inhibit the nucleophilic attack activity. For example, a small-molecule could interact directly with certain amino acids in the site (e.g., the catalytic triad amino acids) to inhibit the postulated reaction mechanism, or could interact at an allosteric site, i.e., a region of the molecule not directly involved the catalytic activity but to which binding of a compound results (e.g., by the induction in a conformational change in the molecule) in inhibition of the activity.

By “molecular modeling” is meant quantitative and/or qualitative analysis of the structure and function of physical interactions based on three-dimensional structural information and interaction models. This includes conventional numeric-based molecular dynamic and energy minimization models, interactive computer graphic models, modified molecular mechanics models, distance geometry and other structure-based constraint models. Molecular modeling typically is performed using a computer and may be further optimized using known methods.

Methods of designing compounds that bind specifically (e.g., with high affinity) to the glutamine formation active site, including one or both of the catalytic triads, typically are also computer-based, and involve the use of a computer having a program capable of generating an atomic model. Computer programs that use X-ray crystallography data are particularly useful for designing such compounds. Programs such as RasMol, for example, can be used to generate a three dimensional model of, e.g., deadenylylated or adenylylated GS, a fragment of deadenylylated or adenylylated GS, or a collection of residues making up all or part of the glutamine formation active site of deadenylylated or adenylylated GS, such as residues making up a catalytic triad active site of deadenylylated or adenylylated GS. Computer programs such as INSIGHT (Accelrys, Burlington, Mass.), Auto-Dock (Accelrys), and Discovery Studio 1.5 (Accelrys) allow for further manipulation and the ability to introduce new structures.

Compounds can be designed using, for example, computer hardware or software, or a combination of both. However, designing is preferably implemented in one or more computer programs executing on one or more programmable computers, each containing a processor and at least one input device. The computer(s) preferably also contain(s) a data storage system (including volatile and non-volatile memory and/or storage elements) and at least one output device. Program code is applied to input data to perform the functions described above and generate output information. The output information is applied to one or more output devices in a known fashion. The computer can be, for example, a personal computer, microcomputer, or work station of conventional design.

Each program is preferably implemented in a high level procedural or object oriented programming language to communicate with a computer system. However, the programs can be implemented in assembly or machine language, if desired. In any case, the language can be a compiled or interpreted language.

Each computer program is preferably stored on a storage media or device (e.g., ROM or magnetic diskette) readable by a general or special purpose programmable computer. The computer program serves to configure and operate the computer to perform the procedures described herein when the program is read by the computer. The method of the invention can also be implemented by means of a computer-readable storage medium, configured with a computer program, where the storage medium so configured causes a computer to operate in a specific and predefined manner to perform the functions described herein.

For example, the computer-requiring steps in a method of designing a test compound can involve:

(a) inputting into an input device, e.g., through a keyboard, a diskette, or a tape, data (e.g. atomic coordinates) that define the three-dimensional (3-D) structure of a first molecule or complex (e.g., adenylylated GS, deadenylylated GS; a portion of deadenylylated GS or adenylylated GS (e.g., a collection of residues making up some or all of the glutamine formation active site, such as residues making up either or both catalytic triads), any of which could include one or more bound ATP, ADP, glutamine, or glutamate molecules) that binds to a second molecule or complex (e.g., ATP, ADP, glutamine, glutamate, γ-glutamyl phosphate); and

(b) determining, using a processor, the 3-D structure (e.g., an atomic model) of the site on the first molecule or complex involved in binding to the second molecule or complex.

It should be noted that although the original GS 3-D structure is taken from one species, one of skill in art could, by standard methods, e.g., homology alignments or molecular modeling, establish the corresponding residues of interest of GS in other species. From the information obtained in this way, one skilled in the art will be able to design and make inhibitory compounds (e.g., peptides, non-peptide small molecules, peptidomimetics, and aptamers (e.g., nucleic acid aptamers)) with the appropriate 3-D structure. For example, one of skill in the art could design inhibitory compounds that could interact with certain residues of the first molecule or complex. Thus, after step (b), one could design, based on the three-dimensional structure, a test compound capable of inhibiting the interaction between, for example, the glutamine formation active site and a y-glutamyl phosphate intermediate, such as a test compound capable of inhibiting the interaction between an adenylylated catalytic triad site of the glutamine formation active site and a γ-glutamyl phosphate intermediate, or of inhibiting the interaction between an deadenylylated catalytic triad site of the glutamine formation active site and a γ-glutamyl phosphate intermediate. In some embodiments, a test compound can be designed that is capable of inhibiting the formation of an acyl-enzyme intermediate in the adenylylated catalytic triad site of the glutamine formation active site, or of inhibiting the formation of the acyl-enzyme intermediate in the deadenylylated catalytic triad site of the glutamine formation active site.

Moreover, if computer-usable 3-D data (e.g., x-ray crystallographic data) for a candidate compound are available, one or more of the following computer-based steps can be performed in conjunction with computer-based steps (a) and (b) described above:

(c) inputting into an input device, e.g., through a keyboard, a diskette, or a tape, data (e.g. atomic coordinates) that define the three-dimensional (3-D) structure of a candidate compound;

(d) determining, using a processor, the 3-D structure (e.g., an atomic model) of the candidate compound;

(e) determining, using the processor, whether the candidate compound binds to the site on the first molecule or complex; and

(f) identifying the candidate compound as a compound that inhibits the interaction between the first and second molecules or complexes.

The method can involve an additional step of outputting to an output device a model of the 3-D structure of the compound. In addition, the 3-D data of candidate compounds can be compared to a computer database of, for example, 3-D structures stored in a data storage system.

In some embodiments, a computer-assisted method of generating a test inhibitor of the glutamine formation active site (e.g., catalytic triad activity) of a glutamine synthetase polypeptide, can include:

(a) inputting on the input device data comprising a structure or substructure (e.g., a catalytic triad) of the glutamine formation active site of GS;

(b) docking into the structure a test inhibitor molecule using the processor; and

(c) determining, based on the docking, whether the test inhibitor molecule would inhibit the glutamine formation active site (e.g., catalytic triad) activity.

Compounds of the invention also may be interactively designed from structural information of the compounds described herein using other structure-based design/modeling techniques (see, e.g., Jackson (1997) Seminars in Oncology 24:L164-172; and Jones et al. (1996) J. Med. Chem. 39:904-917). Compounds and polypeptides of the invention also can be identified by, for example, identifying candidate compounds by computer modeling as fitting spatially and preferentially (i.e., with high affinity) into the glutamine formation active site.

Candidate compounds identified as described above can then be tested in standard cellular or cell-free enzymatic or enzymatic inhibition assays familiar to those skilled in the art. Exemplary assays are described herein.

The 3-D structure of biological macromolecules (e.g., GS, adenylylated GS), nucleic acids, carbohydrates, and lipids) can be determined from data obtained by a variety of methodologies. These methodologies, which have been applied most effectively to the assessment of the 3-D structure of proteins, include: (a) x-ray crystallography; (b) nuclear magnetic resonance (NMR) spectroscopy; (c) analysis of physical distance constraints formed between defined sites on a macromolecule, e.g., intramolecular chemical crosslinks between residues on a protein (e.g., International Patent Application No. PCT/US00/14667, the disclosure of which is incorporated herein by reference in its entirety), and (d) molecular modeling methods based on a knowledge of the primary structure of a protein of interest, e.g., homology modeling techniques, threading algorithms, or ab initio structure modeling using computer programs such as MONSSTER (Modeling Of New Structures from Secondary and Tertiary Restraints) (see, e.g., International Application No. PCT/US99/11913, the disclosure of which is incorporated herein by reference in its entirety). Other molecular modeling techniques may also be employed in accordance with this invention [e.g., Cohen et al. (1990) J. Med. Chem. 33: 883-894; Navia et al (1992) Current Opinions in Structural Biology, 2, pp. 202-210, the disclosures of which are incorporated herein by reference in its entirety]. All these methods produce data that are amenable to computer analysis. Other spectroscopic methods that can also be useful in the method of the invention, but that do not currently provide atomic level structural detail about biomolecules, include circular dichroism and fluorescence and ultraviolet/visible light absorbance spectroscopy. A preferred method of analysis is x-ray crystallography. Descriptions of this procedure and of NMR spectroscopy are provided below.

X-Ray Crystallography

X-ray crystallography is based on the diffraction of x-radiation of a characteristic wavelength by electron clouds surrounding the atomic nuclei in a crystal of the region of interest. The technique uses crystals of purified biological macromolecules (but these frequently include solvent components, co-factors, substrates, or other ligands) to determine near atomic resolution of the atoms making up the particular biological macromolecule. A prerequisite for solving the 3-D structure of the macromolecule by x-ray crystallography is a well-ordered crystal that will diffract x-rays strongly. The method directs a beam of x-rays onto a regular, repeating array of many identical molecules so that the x-rays are diffracted from the array in a pattern from which the structure of an individual molecule can be retrieved. Well-ordered crystals of, for example, globular protein molecules are large, spherical or ellipsoidal objects with irregular surfaces. The crystals contain large channels between the individual molecules. These channels, which normally occupy more than one half the volume of the crystal, are filled with disordered solvent molecules, and the protein molecules are in contact with each other at only a few small regions. This is one reason why structures of proteins in crystals are generally the same as those of proteins in solution.

GS has been crystallized many times, e.g., from Salmonella typhimurium, Almassy, R J. et. al. (1986) Nature (London) 323: 304-309, Liaw, S-H., et al. (1993) Proc. Natl. Acad. Sci. (USA) 90: 4996-5000; with glycine, alanine and serine in the active site, Liaw, S-H and D. Eisenberg (1994) J. Biol. Chem. 33: 675-681; with AMPPNP, glutamate, L-methionine-S-sulfoximine, glutamine and ADP in the active site of 5 structures, Liaw, S-H., Jun, G. and D. Eisenberg (1993) Protein Science, 2: 470-471; and with ATP in the active site, Liaw, S-H., Jun, G and D. Eisenberg (1994) Biochemistry 33: 11184-11188. Methods of obtaining GS or GS fragments, including adenylylated GS, are described below or are well known to those having ordinary skill in the art. The formation of crystals is dependent on a number of different parameters, including pH, temperature, the concentration of the biological macromolecule, the nature of the solvent and precipitant, as well as the presence of added ions or ligands of the protein. Many routine crystallization experiments may be needed to screen all these parameters for the combinations that give a crystal suitable for x-ray diffraction analysis. Crystallization robots can automate and speed up work of reproducibly setting up a large number of crystallization experiments (see, e.g., U.S. Pat. No. 5,790,421).

Polypeptide crystallization occurs in solutions in which the polypeptide concentration exceeds it's solubility maximum (i.e., the polypeptide solution is supersaturated). Such solutions may be restored to equilibrium by reducing the polypeptide concentration, preferably through precipitation of the polypeptide crystals. Often polypeptides may be induced to crystallize from supersaturated solutions by adding agents that alter the polypeptide surface charges or perturb the interaction between the polypeptide and bulk water to promote associations that lead to crystallization.

Crystallizations are generally carried out between 4° C. and 20° C. Substances known as “precipitants” are often used to decrease the solubility of the polypeptide in a concentrated solution by forming an energetically unfavorable precipitating depleted layer around the polypeptide molecules [Weber (1991) Advances in Protein Chemistry, 41:1-36]. In addition to precipitants, other materials are sometimes added to the polypeptide crystallization solution. These include buffers to adjust the pH of the solution and salts to reduce the solubility of the polypeptide. Various precipitants are known in the art and include the following: ethanol, 3-ethyl-2-4 pentanediol, and many of the polyglycols, such as polyethylene glycol (PEG). The precipitating solutions can include, for example, 13-24% PEG 4000, 5-41% ammonium sulfate, and 1.0-1.5 M sodium chloride, and a pH ranging from 5-7.5. Other additives can include 0.1 M Hepes, 2-4% butanol, 0.1 M or 20 mM sodium acetate, 50-70 mM citric acid, 120-130 mM sodium phosphate, 1 mM ethylene diamine tetraacetic acid (EDTA), and 1 mM dithiothreitol (ITT). These agents are prepared in buffers and are added dropwise in various combinations to the crystallization buffer.

Commonly used polypeptide crystallization methods include the following techniques: batch, hanging drop, seed initiation, and dialysis. In each of these methods, it is important to promote continued crystallization after nucleation by maintaining a supersaturated solution. In the batch method, polypeptide is mixed with precipitants to achieve supersaturation, and the vessel is sealed and set aside until crystals appear. In the dialysis method, polypeptide is retained in a sealed dialysis membrane that is placed into a solution containing precipitant. Equilibration across the membrane increases the polypeptide and precipitant concentrations, thereby causing the polypeptide to reach supersaturation levels.

In the preferred hanging drop technique [McPherson (1976) J. Biol. Chem., 251:6300-6306], an initial polypeptide mixture is created by adding a precipitant to a concentrated polypeptide solution. The concentrations of the polypeptide and precipitants are such that in this initial form, the polypeptide does not crystallize. A small drop of this mixture is placed on a glass slide that is inverted and suspended over a reservoir of a second solution. The system is then sealed. Typically, the second solution contains a higher concentration of precipitant or other dehydrating agent. The difference in the precipitant concentrations causes the protein solution to have a higher vapor pressure than the second solution. Since the system containing the two solutions is sealed, an equilibrium is established, and water from the polypeptide mixture transfers to the second solution. This equilibrium increases the polypeptide and precipitant concentration in the polypeptide solution. At the critical concentration of polypeptide and precipitant, a crystal of the polypeptide may form.

Another method of crystallization introduces a nucleation site into a concentrated polypeptide solution. Generally, a concentrated polypeptide solution is prepared and a seed crystal of the polypeptide is introduced into this solution. If the concentrations of the polypeptide and any precipitants are correct, the seed crystal will provide a nucleation site around which a larger crystal forms.

Yet another method of crystallization is an electrocrystallization method in which use is made of the dipole moments of protein macromolecules that self-align in the Helmholtz layer adjacent to an electrode (see U.S. Pat. No. 5,597,457).

Some proteins may be recalcitrant to crystallization. However, several techniques are available to the skilled artisan to induce crystallization. For example, the removal of flexible polypeptide segments at the amino or carboxyl terminal end of the protein may facilitate production of crystalline protein samples. Removal of such segments can be done using molecular biology techniques or treatment of the protein with proteases such as trypsin, chymotrypsin, or subtilisin.

In diffraction experiments, a narrow and parallel beam of x-rays is taken from the x-ray source and directed onto the crystal to produce diffracted beams. The incident primary beams cause damage to both the macromolecule and solvent molecules. The crystal is, therefore, cooled (e.g., to −220° C. to −50° C.) to prolong its lifetime. The primary beam must strike the crystal from many directions to produce all possible diffraction spots, so the crystal is rotated in the beam during the experiment. The diffracted spots are recorded on a film or by an electronic detector. Exposed film has to be digitized and quantified in a scanning device, whereas the electronic detectors feed the signals they detect directly into a computer. Electronic area detectors significantly reduce the time required to collect and measure diffraction data. Each diffraction beam, which is recorded as a spot on film, is defined by three properties: the amplitude, which is measured from the intensity of the spot; the wavelength, which is set by the x-ray source; and the phase, which is lost in x-ray experiments. All three properties are needed for all of the diffracted beams in order to determine the positions of the atoms giving rise to the diffracted beams. One way of determining the phases is called Multiple Isomorphous Replacement (MIR), which requires the introduction of exogenous x-ray scatterers (e.g., heavy atoms such metal atoms) into the unit cell of the crystal. For a more detailed description of MIR, see U.S. Pat. No. 6,093,573, column 15.

Atomic coordinates refer to Cartesian coordinates (x, y, and z positions) derived from mathematical equations involving Fourier synthesis of data derived from patterns obtained via diffraction of a monochromatic beam of x-rays by the atoms (scattering centers) of biological macromolecule of interest in crystal form. Diffraction data are used to calculate electron density maps of repeating units in the crystal (unit cell). Electron density maps are used to establish the positions (atomic coordinates) of individual atoms within a crystal's unit cell. The absolute values of atomic coordinates convey spatial relationships between atoms because the absolute values ascribed to atomic coordinates can be changed by rotational and/or translational movement along x, y, and/or z axes, together or separately, while maintaining the same relative spatial relationships among atoms. Thus, a biological macromolecule (e.g., a protein) whose set of absolute atomic coordinate values can be rotationally or translationally adjusted to coincide with a set of prior determined values from an analysis of another sample is considered to have the same atomic coordinates as those obtained from the other sample.

Further details on x-ray crystallography can be obtained from U.S. Pat. No. 6,093,573 and International Application Nos. PCT/US99/18441, PCT/US99/11913, and PCT/US00/03745.

NMR Spectroscopy

While x-ray crystallography requires single crystals of a macromolecule of interest, NMR measurements are carried out in solution under near physiological conditions. However, NMR-derived structures are not as detailed as crystal-derived structures.

While the use of NMR spectroscopy was until relatively recently limited to the elucidation of the 3-D structure of relatively small molecules (e.g., proteins of 100-150 amino acid residues), recent advances including isotopic labeling of the molecule of interest and transverse relaxation-optimized spectroscopy (TROSY) have allowed the methodology to be extended to the analysis of much larger molecules, e.g., proteins with a molecular weight of 110 kDa [Wider (2000) BioTechniques, 29:1278-1294].

NMR uses radio-frequency radiation to examine the environment of magnetic atomic nuclei in a homogeneous magnetic field pulsed with a specific radio frequency. The pulses perturb the nuclear magnetization of those atoms with nuclei of nonzero spin. Transient time domain signals are detected as the system returns to equilibrium. Fourier transformation of the transient signal into a frequency domain yields a one-dimensional NMR spectrum. Peaks in these spectra represent chemical shifts of the various active nuclei. The chemical shift of an atom is determined by its local electronic environment. Two-dimensional NMR experiments can provide information about the proximity of various atoms in the structure and in three dimensional space. Protein structures can be determined by performing a number of two- (and sometimes 3- or 4-) dimensional NMR experiments and using the resulting information as constraints in a series of protein folding simulations.

More information on NMR spectroscopy including detailed descriptions of how raw data obtained from an NMR experiment can be used to determine the 3-D structure of a macromolecule can be found in: Protein NMR Spectroscopy, Principles and Practice,

J. Cavanagh et al., Academic Press, San Diego, 1996; Gronenbom et al. (1990) Anal. Chem. 62(1):2-15; and Wider (2000), supra.

Any available method can be used to construct a 3-D model of a GS region of interest from the x-ray crystallographic and/or NMR data using a computer as described above. Such a model can be constructed from analytical data points inputted into the computer by an input device and by means of a processor using known software packages, e.g., CATALYST (Accelrys), INSIGHT (Accelrys) and CeriusII, HKL, MOSFILM, XDS, CCP4, SHARP, PHASES, HEAVY, XPLOR, TNT, NMRCOMPASS, NPIPE, DIANA, NMRDRAW, FELIX, VNMR, MADIGRAS, QUANTA, BUSTER, SOLVE, O, FRODO, or CHAIN. The model constructed from these data can be visualized via an output device of a computer, using available systems, e.g., Silicon Graphics, Evans and Sutherland, SUN, Hewlett Packard, Apple Macintosh, DEC, IBM, or Compaq.

### Designing Compounds of the Invention

Once the 3-D structure of a compound that binds to and/or inhibits a GS region of interest (e.g., an adenylylated or deadenylylated GS glutamine formation active site, including an adenylylated or deadenylylated catalytic triad site) has been established using any of the above methods, a compound that has substantially the same 3-D structure (or contains a domain that has substantially the same structure) as the identified compound can be made. In this context, “has substantially the same 3-D structure” means that the compound means that the compound possesses a hydrogen bonding region and hydrophobic character that is similar to the identified compound. In some cases, a compound having substantially the same 3-D structure as the identified compound can include possesses a hydrogen bonding zone which is similar in structure to and charge distribution of γ-glutamyl phosphate, or capable of forming an acyl-enzyme intermediate with either Ser 52 or Ser 53 (or the corresponding residues on GS from a species other than E. coli).

With the above described 3-D structural data in hand and knowing the chemical structure (e.g., amino acid sequence in the case of a protein) of the region of interest, those of skill in the art would know how to make compounds with the above-described properties. Such methods include chemical synthetic methods and, in the case of proteins, recombinant methods (see above). For example, cysteine residues appropriately placed in a compound so as to form disulfide bonds can be used to constrain the compound or a domain of the compound in an appropriate 3-D structure. In addition, in a compound that is a polypeptide or includes a domain that is a polypeptide, one of skill in the art would know what amino acids to include and in what sequence to include them in order to generate, for example, α-helices, β structures, or sharp turns or bends in the polypeptide backbone.

While not essential, computer-based methods can be used to design the compounds of the invention. Appropriate computer programs include: InsightII (Accelrys), CATALYST (Accelrys), LUDI (Accelrys., San Diego, Calif.), Aladdin (Daylight Chemical Information Systems, Irvine, Calif.); and LEGEND [Nishibata et al. (1985) J. Med. Chem. 36(20):2921-2928].

Compounds of the invention that are peptides also include those described above, but modified for in vivo use by the addition, at the amino- and/or carboxyl-terminal ends, of a blocking agent to facilitate survival of the relevant polypeptide in viva. This can be useful in those situations in which the peptide termini tend to be degraded by proteases prior to cellular uptake. Such blocking agents can include, without limitation, additional related or unrelated peptide sequences that can be attached to the amino and/or carboxyl terminal residues of the peptide to be administered. This can be done either chemically during the synthesis of the peptide or by recombinant DNA technology by methods familiar to artisans of average sdill.

Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Likewise, the peptide compounds can be covalently or noncovalently coupled to pharmaceutically acceptable “carrier” proteins prior to administration.

Also of interest are peptidomimetic compounds that are designed based upon the amino acid sequences of compounds of the invention that are peptides. Peptidomimetic compounds are synthetic compounds having a three-dimensional conformation (i.e., a “peptide motif”) that is substantially the same as the three-dimensional conformation of a selected peptide. Peptidomimetic compounds can have additional characteristics that enhance their in vivo utility, such as increased cell permeability and prolonged biological half-life.

The peptidomimetics typically have a backbone that is partially or completely non-peptide, but with side groups that are identical to the side groups of the amino acid residues that occur in the peptide on which the peptidomimetic is based. Several types of chemical bonds, e.g., ester, thioester, thioamide, retroamide, reduced carbonyl, dimethylene and ketomethylene bonds, are known in the art to be generally useful substitutes for peptide bonds in the construction of protease-resistant peptidomimetics.

Given the proposed switch between catalytic triads depending on the adenylylation state of GS, small-molecule, peptidic, or peptidomimetic compounds that are known to be or postulated to be protease inhibitors, particularly serine protease inhibitors, or analogues of such protease inhibitors, are of particular interest. Such molecules can be used in the computer-based methods described herein, e.g., as molecules to dock into, e.g., a glutamine formation active site or catalytic triad site, in order to design other test inhibitor molecules, or can be used in the therapeutic or in vivo or in vitro inhibition methods described below. Exemplary compounds include those disclosed in WO 99/12935; WO 02/22575; WO 97/46576; WO 99/50263; and WO 99/50257. Known serine protease inhibitors include peptidyl-arginine aldehyde derivatives, pyrrolopyrrrolidene derivatives, quinolinone derivatives, and 1-methyl benzimidazole derivatives.

Screening Assays

Provided herein are in vitro methods for identifying compounds that inhibit GS activity, such as by inhibiting a glutamine formation active site of adenylylated and/or deadenylylated GS, and/or by inhibiting either or both of the adenylylated or deadenylylated catalytic triads' activities.

The specificity of inhibition of adenylylated GS can differ from that of the deadenylylated GS because of differences in the structures of the active catalytic triads. In methods of screening for compounds that inhibit the glutamine formation active site of GS, including compounds that inhibit either or both of the catalytic triads, assays can be designed to screen for compounds that inhibit nucleophilic attack by ammonia on the γ-glutamyl phosphate intermediate produced in the first step of the GS enzymatic reaction mechanism; that inhibit the attack of an activated serine of a catalytic triad on the γ-glutamyl phosphate in the formation of a glutamyl acyl-enzyme intermediate; or that inhibit nucleophilic attack by ammonia on the glutamyl-acyl intermediate. For example, in one assay, a GS polypeptide can be contacted with a test compound under specific assay conditions effective for glutamine formation to occur. Typically glutamine synthetase polypeptide is tested with the inhibitor under conditions for the adenylylated form of the enzyme at concentrations of 0.6 mM ATP, 1.8 mM MnCl₂, 7.2 mM NaHCO₃, 4 mM glutamic acid, and 4 mM NH₄Cl in 10 mM Imidazole.HCl buffer (pH 6.3); and for the deadenylylated form of the assay 0.6 mM ATP, 0.6 mM MgCl₂, 4 mM glutamic acid, 4 NH₄Cl and 10 mM Imidazole.HCl buffer (pH 7.2) All assays were run at 37° C.

Assays for adenylylated GS can be different from those for deadenylylated GS. The adenylylated GS assay can be run at pH 6.3 and a HCO₃— to Mn²⁺ to ATP concentration ratio of 12:3:1, while the deadenylylated GS assay can be run at pH 7.2 and a Mg²⁺ to ATP concentration ratio of 1:1. Typical assay conditions for the adenylylated GS are 20 mM Imidazole buffer (pH 6.3), 1 mM ATP, 3 mM MnCl₂, 12 mM NaHCO₃ and 2 mM sodium glutamate; typical assay conditions for deadenylylated GS are 20 mM Imidazole buffer (pH 7.2), 1 mM ATP, 1 mM MgCl₂, 12 mM NaCl and 2 mM sodium glutamate. Assays can be run at 37° C.

ATP hydrolysis, ADP production and/or glutamate utilization and glutamine production can be measured in the presence and absence of the test compound. For example, in one embodiment, a method of screening a test compound in vitro to determine whether or not it inhibits the glutamine formation active site activity of a glutamine synthetase polypeptide includes:

(a) contacting a glutamine synthetase polypeptide (e.g., an adenylylated GS or an unadenylylated GS), with a test compound under conditions effective for glutamine formation active site activity to occur; and

(b) determining whether or not the glutamine formation active site activity of the glutamine synthetase polypeptide is reduced relative to the activity of a glutamine synthetase polypeptide that has not been contacted with the test compound.

The glutamine formation active site activity can be mediated by a glutamyl acyl-enzyme intermediate, as described herein. If an adenylylated GS polypeptide is used, any inhibitory activity can be compared with the inhibition obtained for the test compound on a deadenylylated glutamine synthetase, and vice versa.

### Compounds and Pharmaceutical Compositions

Provided herein also are compounds, e.g., compounds for inclusion in pharmaceutical compositions and/or for use in the methods described herein. Based on structural and mechanistic information on the glutamine formation active site of GS, as demonstrated herein and in the Examples below, known protease inhibitors, including known serine protease inhibitors, can be screened for their GS inhibitory activity, as described above. In some embodiments, compounds to be evaluated for protease inhibition activity, also referred to as “test” protease inhibitors herein, can be similarly screened. For example, a library of test protease inhibitors can be screened, such as a library of test serine protease inhibitors. Compounds having appropriate inhibitory activity can then be used to treat, prevent, or ameliorate one or more symptoms associated with bacterial infections in mammals. Useful serine protease inhibitors are described in WO 99/12935; WO 02/22575; WO 97/46576; WO 99/50263; and WO 99/50257.

### Formulation of Pharmaceutical Compositions

A pharmaceutical composition provided herein contains therapeutically effective amounts of one or more compounds, e.g., serine protease inhibitors, that are useful in the treatment, prevention, or amelioration of one or more of the symptoms associated with a bacterial infection (e.g., a bacteria containing the GSI-β gene, such as Mycobacterium tuberculosis, or a bacteria containing the GSI-α gene, such as Bacillus anthracis), or a disorder, condition, or ailment in which such a bacterial infection is implicated, and a pharmaceutically acceptable carrier. Pharmaceutical carriers suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. For example, the compounds may be formulated or combined with known antibacterial compounds, anti-inflammatory compounds, steroids, and/or antivirals.

The compounds are, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives thereof is (are) mixed with a suitable pharmaceutical carrier. The compounds may be derivatized as the corresponding salts, esters, enol ethers or esters, acetals, ketals, orthoesters, hemiacetals, hemiketals, acids, bases, solvates, hydrates or prodrugs prior to formulation, as described above. The concentrations of the compounds in the compositions are effective for delivery of an amount, upon administration, that treats, prevents, or ameliorates one or more of the symptoms of a bacterial infection.

In one embodiment, the compositions are formulated for single dosage administration. To formulate a composition, the weight fraction of compound is dissolved, suspended, dispersed or otherwise mixed in a selected carrier at an effective concentration such that the treated condition is relieved or one or more symptoms are ameliorated.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro, ex vivo and in vivo systems, and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art.

Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The active ingredient may be administered at once, or may be divided into a number of smaller doses to be administered at intervals of time. It is understood that the precise dosage and duration of treatment is a function of the disorder being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, or in one embodiment 0.1-95%.

### Articles of Manufacture

A compound or pharmaceutically acceptable derivative may be packaged as an article of manufacture (e.g., lit) containing packaging material, a compound or pharmaceutically acceptable derivative thereof provided herein within the packaging material, and a label that indicates that the compound or composition, or pharmaceutically acceptable derivative thereof, is useful for treatment, prevention, or amelioration of one or more symptoms or disorders in which a bacterial infection, including a Mycobacterium tuberculosis infection, is implicated.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment.

### Evaluation of the Activity of the Compounds

The activity of the compounds provided herein as inhibitors of GS activity, e.g., adenylylated GS activity, deadenylylated GS activity, glutamine formation active site activity, or one or more catalytic triad activities; and/or as compounds to treat, prevent, or ameliorate one or more symptoms, conditions, or disorders associated with a bacterial infection (e.g., Mycobacterium tuberculosis infection), may be measured or evaluated in standard assays. Enzymatic inhibition assays (e.g., γ-glutamyl transferase assays, ATP hydrolysis assays, ADP production, and glutamate utilization and glutamine formation assays), inhibition of growth of a bacteria such as M. tuberculosis, and cell cytoprotection, viability, and cytotoxicity assays, all of which are well known to those having ordinary skill in the art and/or are described below.

### Methods of Use of the Compounds and Compositions

Both in vitro or in vivo methods can be performed with the compounds and compositions described herein. In vitro application of the compounds of the invention can be useful, for example, in basic scientific studies of GS reaction mechanisms, or for in vitro methods of treating, preventing, reducing, or inhibiting a bacterial contamination or infection, or for inhibiting a glutamine formation active site of GS. The compounds can also be used in vivo as therapeutic agents against bacterial infections, including pathogenic or opportunistic bacteria. In particular, the compounds can be used as therapeutic agents against infections from GSI-β bacteria such as Corynebacterium diphtheriae, Neisseria gonorrhoeae, Escherichia coli, Salmonella typhinurium, Salmonella typhi, Klebsiella pneumoniae, Serratia marcescens, Proteus vulgaris, Shigella dysenteriae, Vibrio cholerae, Pseudomonas aeruginosa, Alcaligenes faecalis, Helicobacter pylori, Haemophilus influenzae, Bordetella pertussis, Bordella bronchiseptia, Neisseria meningitides, Brucella melitensis, Mycobacterium tuberculosis, Mycobacterium leprae, Treponema pallidum, Leptospira interrogans, Actinomyces israelii, Nocardia esteroides, Thiobacillus ferrooxidans, Azospirillum brazilensis, Anabaena sp., Fremyella diplosiphon, and Streptomyces coelicolor, or infections from GSI-α bacteria such as Bacillus cereus, Bacillus subtilis, Bacillus anthracis, Streptococcus pneumoniae, Streptococcus pyogenes, Staphylococcus aereus, Clostridium botulinum, Clostridium tetani and Clostridium perfringens. The compounds can be useful in the prevention and/or therapy of diseases involving intracellular microorganisms (i.e., infectious agents that replicate inside a cell), e.g., intracellular bacteria such as M. tuberculosis.

The methods of the invention can be applied to a wide range of species, e.g., mammals such as humans, non-human primates (e.g., monkeys), horses, cattle, pigs, sheep, goats, dogs, cats, rabbits, guinea pigs, hamsters, rats, and mice. As bacterial GSI-β enzymes are regulated via the adenylylation/deadenylylation cascade, but mammalian GSII enzymes are not, and as certain of the inhibitors herein are postulated to target adenylylated GS selectively, the compounds and compositions can be used to selectively inhibit bacterial cell growth while minimally negatively impacting mammalian cells.

In one in vivo approach, a compound or pharmaceutical composition described herein can be administered to the subject, e.g., a mammal, such as a mammal suspected of suffering from a bacterial infection or suffering from a bacterial infection. Generally, the compounds of the invention will be suspended in a pharmaceutically-acceptable carrier (e.g., physiological saline) and administered orally or transdermally or injected (or infused) intravenously, subcutaneously, intramuscularly, intraperitoneally, intrarectally, intravaginally, intranasally, intragastrically, intratracheally, or intrapulmonarily. They can be delivered directly to an appropriate affected tissue.

The dosages of the inhibitory compounds and supplementary agents to be used depend on the choice of the route of administration; the nature of the formulation; the nature of the patient's illness; the subject's size, weight, surface area, age, and sex; other drugs being administered; and the judgment of the attending physician. Suitable dosages are generally in the range of 0.0001-100.0 mg/kg. Wide variations in the needed dosage are to be expected in view of the variety of compounds and supplementary agents available and the differing efficiencies of various routes of administration. For example, oral administration would be expected to require higher dosages than administration by i.v. injection. Variations in these dosage levels can be adjusted using standard empirical routines for optimization as is well understood in the art. Administrations of compounds and/or supplementary agents can be single or multiple (e.g., 2-, 3-, 4-, 6-, 8-, 10-, 20-, 50-, 100-, 150-, or more fold).

### EXAMPLES

### Example 1

### Identification of Catalytic Triads from Structural and Molecular Dynamics Studies

Structural and molecular dynamics analysis of the active site of the glutamine synthetase from E. coli led to the identification of two protease-like catalytic triads. A DNAMAN sequence alignment of GS sequences from both GS1-α and GS1-β species indicated that the putative amino acid residues making up the catalytic triad are conserved throughout all species. Energy minimizations using DISCOVER_{—}3 from the INSIGHTII suite of software were then used to try to group the amino acids making up each active site and confirm the findings from site directed mutatgenesis. The energy minimizations were carried out on GS molecules that had been reduced to a dimer to ensure that a full active site was obtained, but to reduce the total number of atoms to facilitate computation.

The high degree of conservation in the GS protein sequences of prokaryotic microorganisms facilitated a molecular modelling exercise of the glutamine synthetase of E. coli. The crystal structure model used was 1f52.pdb (Gill H S and D Eisenberg (2001) Biochemistry 7: 1903-1912), which was obtained from the Brookhaven Protein Database. The dimer was also manually adenylylated.

Within the active site of glutamine synthetase, which occurs at the interface of two subunits, a cluster of serine and histidine residues were found. These residues are Ser52, Ser53, His210 and His211 (using the E. coli residue numbering). A number of acid residues, Asp50, Glu129, Glu327 and Glu357, required to complete the putative catalytic triads, were also located in the active site.

Thus, the proposed catalytic triads are believed to include the following residues from two separate subunits (designated A and B):
Adenylylated GS:
   Ser52 (Subunit B)
   His211 (Subunit A)
   Glu129 (Subunit A)
Deadenylylated GS:
   Ser53 (Subunit B)
   His210 (Subunit A)
   Glu357 (Subunit A)

As one of skill in the art will recognize, the corresponding residues of GS from other species than E. coli could be determined using a number of known techniques, e.g., molecular modelling and/or homology alignments, as well as knowledge of the proposed catalytic triad mechanism.

### Example 2

### Site-Directed Mutagenesis and Mutant Gene Expression

Molecular modelling of the active site of the glutamine synthetase from E. coli led to the identification of two protease-like catalytic triads. In order to investigate the role that these residues played with respect to the regulation of the glutamine synthetase from E. coli, site-directed mutagenesis (SDM) was used to specifically target these residues and then to assess the effect that altering these residues had on the catalytic activities of glutamine synthetase.

Two different systems were used for the SDM. The first was the Altered Sites™ II in vitro Mutagenesis System from Promega, and the second was the QuikChange™ XL Site-Directed Mutagenesis System from Stratagene. The majority of mutations were carried out using the Altered Sites™ System, as it provides a high-efficiency procedure for the generation and selection of oligonucleotide-directed mutants. The system allows for the mutagenesis of double-stranded template DNA, as well as for sequential rounds of mutagenesis without any need for subcloning. The procedure uses antibiotic selection as a means to obtain a high frequency of mutants. The vector contains two antibiotic resistance markers: a tetracycline resistance marker, which is active and an ampicillin resistance marker, which is inactive. Oligonucleotides which restore and knockout the two markers are provided in the kit. During the first round of mutagenesis, the tetracycline resistance gene is inactivated and the ampicillin resistance is restored. Should a second round of mutagenesis need be carried out on the mutant generated in the first round of mutagenesis, then the ampicillin resistance gene is again inactivated and the tetracycline resistance restored. Thus, multiple rounds of mutagenesis are very easily carried out, and the yield of mutants is increased.

Because a few mutations could not be achieved using this system, the QuikChange™ System from Stratagene was used, which is a polymerase chain reaction-based procedure that can be used to introduce mutations into virtually any vector. The basic procedure utilises a supercoiled double-stranded vector containing the insert of interest and two synthetic complementary oligonucleotide primers containing the desired mutation. The primers, each complementary to opposite strands of the vector, are extended during temperature cycling using PfuTurbo DNA polymerase. Incorporation of the oligonucleotide primers generates a mutated plasmid containing staggered nicks. Following temperature cycling, the product is treated with Dpn I. This restriction endonuclease is specific for methylated and hemi-methylated DNA and digests the parental DNA template, thereby selecting for mutation-containing synthesised DNA. The nicked vector DNA incorporating the desired mutations is then transformed into an E. coli host strain, following which individual colonies can be screened for the mutation.

Silent mutations were created in all of the oligonucleotide primers designed, allowing for the incorporation of a restriction endonuclease site to facilitate screening for mutant genes. This enabled simple, quick screening of a number of the colonies obtained for each mutagenesis reaction without the need for sequencing each one. Once the mutagenesis was complete, each mutant gene was expressed in an E. coli glutamine synthetase auxotroph.

As the catalytic activity of glutamine synthetase is regulated by the covalent addition of an AMP group to a tyrosine residue in each subunit of the enzyme, it was proposed to first alter the tyrosine 397 residue (the site of adenylylation) to valine 397 in order to create an artificially deadenylylated form of the enzyme. Each mutation could then be examined in both the adenylylated and deadenylylated forms of the enzyme. A summary of the other targeted residues, with the changes, is shown in Table 1.

TABLE 1
Summary of the amino acid changes produced to demonstrate the
presence of the catalytic triad.
Catalytic Triad Residues

| Key Residue | Mutation |
|---|---|
| Ser 52 | Ala 52 |
| Ser 53 | Ala 53 |
| His 210 | Val 210 |
| His 211 | Val 211 |
| Asp 50 | Ala 50 |
| Glu 129 | Ala 129 |
| Glu328 | Val 328 |
| Glu 357 | Ala 357 |

Materials and Methods

All E. coli cultures were maintained on LM medium (5 g/l NaCl, 10 μl yeast extract, 10 g/l tryptone; pH 7.2) unless otherwise stated. Agar was added at a concentration of 15 g/l when required. The medium was supplemented with 50 μg/ml ampicillin or 12.5 μg/ml tetracycline for pAlter-1, and with 100 μg/ml ampicillin for pBluescript II SK⁺.

The strains and plasmids used in this study are listed in Table 2.

TABLE 2
Bacterial Strains and Plasmids Used

| | | |
|---|---|---|
| Plasmids/ | | |
| Strains | Description | Properties |

Plasmids:

| | | |
|---|---|---|
| pAlter-1 | Site-directed mutagenesis | |
| | vector - Tet^{R}; Amp^{S} | |
| | (Promega) | |
| pBluescipt II | High copy number vector | |
| SK⁺ | for general cloning and | |
| | expression (Stratagene) | |
| pGln6 | Construct containing the | Backman et al (1981). PNAS 78: |
| | E. coli wild type glnA | 3743-3747. |
| | gene | |

Strains:

| | | |
|---|---|---|
| E. coli | Host strain | endA1 recA1 gyrA96 thi hsdR17 |
| JM109 | | (rₖ⁻,mₖ₊) relA1 supE44 λ-Δ(lac- |
| | | proAB) [F′,traD36 proA+B+ |
| | | laclqZΔM15] |
| E. coli XL1- | Host strain | (mcrA)18 (mcrCB-hsdSMR- |
| Blue MR | | mrr)173 endA1 supE44 thi-1 |
| | | recA1 gyrA96 relA1 lac |
| E. coli | Glutamine synthetase | endA thi-1 hsdR17 supE44 |
| YMC 11 | auxotrophic strain | ΔlacU169 hutC_{Klebs} Δ(glnA- |
| | | glnG)2000 |

DNA was isolated on a small scale using either the QiaPrep Spin Miniprep Kit (Qiagen) or by alkaline lysis (Sambrook, J., Fritsch, E. F. and T. Maniatis (1989) In: Molecular Cloning: A Laboratory Manual., 2^{nd} ed. Cold Spring Habor Laboratory Press). Large-scale DNA isolations were performed using the Qiagen Midi DNA Isolation Kit (Qiagen). Digestion of DNA was carried out using restriction enzymes purchased from Amersham Biosciences and used according to the manufacturer's instructions. Alkaline phosphatase was obtained from Amersham Biosciences. T4 DNA Ligase was obtained from Promega and used as per the protocol supplied with the enzyme. Agarose used for electrophoresis of DNA was of molecular biology grade.

Taq polymerase (rTaq) was obtained from TaKaRa Bio Inc. and was used for general screening purposes. High fidelity Taq polymerase (ExTaq) was also obtained from TaKaRa Bio and was used for amplifying genes for cloning.

Restriction digestion and agarose gel elecrophoresis were carried out using standard procedures (Sambrook, J., Fritsch, E. F. and T. Maniatis (1989) In: Molecular Cloning: A Laboratory Manual., 2^{nd} ed. Cold Spring Habor Laboratory Press 1989). A 1 kb DNA ladder was used for all electrophoresis.

Site-directed mutagenesis was carried out using the Altered Sites™ in vitro Mutagenesis Kit from Promega Corporation, or the QuikChange™ XL Site-Directed Mutagenesis Kit from Stratagene, as per the protocols supplied with each kit.

All chemicals were of analytical or molecular biology grade and were used without further purification. All chemicals were obtained from Merck or Sigma unless otherwise stated.

Cloning of the E. coli Glutamine Synthetase Gene

Primers were designed to the sequence of the E. coli glnA gene obtained from Genbank (Accession Number X05173). The primers were designed with NsiI restriction sites (shown in bold) at the 5′ ends. The primers are shown below:

| | |
|---|---|
| 5′ primer: | |
| 5′-GATATGCATCCGTCAAATGCG-3′ | (SEQ ID NO: 1) |
| 3′ primer: | |
| 5′-GCGATGCATAAAGTTTCCACGG-3′ | (SEQ ID NO: 2) |

PCR was performed using DNA of pGLn6 as the template and the above primers. The PCR mixture contained 1 μl of plasmid DNA (50 ng), 5 μl of each primer (2.5 pmol/μl), 4 μl of 2.5 mM dNTP's, 5 μl 10× buffer containing 20 mM MgCl₂ and 0.5 μl of High Fidelity Taq polymerase (2.5 units).

PCR was conducted with the initial denaturation of the template DNA at 95° C. for 5 minutes, followed by 30 cycles of denaturation at 95° C. for 5 minutes, annealing at 55° C. for 1 minute and elongation at 72° C. for 2 minutes. A final elongation step of 72° C. for 10 minutes was also incorporated into the profile. Agarose gel electrophoresis was carried out to verify the fragment size produced by PCR. The PCR product was purified using the HighPure PCR Purification Kit (Roche Diagnostics), and subjected to digestion with NsiI.

To construct the template for SDM with the Altered Sites System, pAlter-1 was linearised with PstI and dephosphorylated prior to ligation. Insert and vector were ligated at an insert:vector ratio of 3:1.

The ligation reaction was transformed into E. coli JM109 by electroporation using a Bio-Rad Gene Pulser, as per the manufacturer's instructions. Transformants were selected on LM Agar supplemented with 12.5 μg/ml tetracycline, 80 μg/ml X-Gal and 1 mM IPTG.

Several white colonies obtained on the transformation plates were then screened by isolating DNA using alkaline lysis, followed by digestion and agarose gel electrophoresis. Sequencing was carried out to confirm the presence and sequence of the E. coli glnA gene. In addition to the use of the M13/F and M13/R universal primers, gene-specific internal primers were designed from the known sequence of the gene. These are shown in Table 3.

TABLE 3
Sequence-specific primers used for
sequencing of the cloned E. coli glnA gene

| Primer | Primer Sequence |
|---|---|
| GlnSeq 1 | 5′-GCTGAACACGTACTGACGATG-3′ |
| | (SEQ ID NO: 3) |
| GlnSeq 2 | 5′-GTGGGAACCGGAGATAGATGATC-3′ |
| | (SEQ ID NO: 4) |
| GlnSeq 3 | 5′-CGATGTTCGGTGATAACGGCTC-3′ |
| | (SEQ ID NO: 5) |
| GlnSeq 4 | 5′-CGTACTTCGATACGACGTGCTTTC-3′ |
| | (SEQ ID NO: 6) |

To construct the template for SDM with the QuikChange™ System, the glutamine synthetase gene was subcloned from the pAlter construct as a SacI-HindIII fragment. The band containing the glnA gene was excised from the gel using a scalpel blade, and pushed through a 2 ml syringe into an Eppendorf tube, to crush the agarose. 1 ml of phenol (equilibrated to pH 8.0) was added to the tube and the suspension was then vortexed for 1 minute. The sample was frozen at −70° C. for at least 30 minutes. Once the sample had thawed, it was centrifuged at 13 000 rpm in an Eppendorf microfuge for 15 minutes. The aqueous phase was removed to a clean tube and then extracted once with phenol:chloroform:isoamyl alcohol (25:24:1), and then once with chloroform:isoamyl alcohol (24:1). The DNA was ethanol precipitated and resuspended in TE Buffer. This fragment was then ligated into pBluescript II SK⁺, also digested with SacI and HindIII, at an insert to vector ratio of 3:1. The ligation reaction was transformed into E. coli XL1-Blue by electroporation, and plated on LM agar plates containing 100 μg/ml ampicillin. Single transformant colonies were screened by isolating plasmid DNA using alkaline lysis, digesting the DNA with BamHI and subsequently analysing the fragments by agarose gel electrophoresis.

Site-Directed Mutagenesis (SDM)

DNA of the wild type glnA gene in pAlter-1 was isolated from E. coli JM109 using the Qiagen Midi Prep Kit. The oligonucleotides designed to carry out the mutagenesis of the glnA gene using this system are listed in Table 4. Silent mutations incorporating a restriction site to facilitate primary screening for the mutation were built in to the SDM oligonucleotides. These are also shown in the Table 4.

TABLE 4
Mutations carried out on the E. coli glnA
gene using the Altered Sites™ System.

| | | Restriction |
|---|---|---|
| Mutation | Oligonucleotide Sequence | Site |
| Y397V | 5′-TGGACAAAAACCTGGTCGACCTGCCGCC | SalI |
| | AG-3′ | |
| | (SEQ ID NO: 7) | |
| S52A | 5′-AAATGTTTGACGGCGCATCGATTGGCGG | ClaI |
| | CTG-3′ | |
| | (SEQ ID NO: 8) | |
| S53A | 5′-GTTTGACGGATCCGCTATTGGCGGCTG | BamHI |
| | G-3′ | |
| | (SEQ ID NO: 9) | |
| S52A | 5′-GTTTGACGGCGCAGCGATCGGCGGCT | PvuI |
| | G-3′ | |
| S53A | (SEQ ID NO: 10) | |
| H210V | 5′-GGTTGAAGCCCATGTGCACGAAGTCGCG | NruI |
| | AGTG-3′ | |
| | (SEQ ID NO: 11) | |
| H211V | 5′-GAAGCCCATCACGTCGAAGTAGCGACAG | PvuII |
| | CTGG-3′ | |
| | (SEQ ID NO: 12) | |
| E327A | 5′-GGTCCCGGGCTATGTAGCATCGATAATG | ClaI |
| | CTG-3′ | |
| | (SEQ ID NO: 13) | |

Mutations to change amino acid residues are shown in bold and restriction sites are underlined.

Following mutagenesis, single colonies were screened for the mutation of choice by isolating DNA from an overnight culture, and performing restriction analysis with the enzyme for the particular mutation being screened for.

Mutant Gene Expression

Mutant genes were isolated from the pAlter-1 clones by digestion with SacI and HindIII. The digests were then subjected to agarose gel electrophoresis to separate the vector and insert bands. The band containing the genes was excised from the gel and the DNA extracted using phenol as described above.

pBluescript II SK⁺ was digested with SacI and HindIII, and each mutant gene was then ligated into this vector at an insert:vector ratio of 3:1. The ligation reaction was transformed into the glutamine synthetase auxotrophic strain E. coli YMC11 by electroporation and transformants were selected on LM Agar supplemented with 50 μg/ml ampicillin.

Single colonies obtained on the transformation plates were subjected to a screening procedure using PCR using the M13/F and M13/R universal primers. In this procedure, single colonies were resuspended in 20 μl of distilled water. 1 μl of this colony suspension was added to a PCR reaction containing 2.5 μl of each primer (2.5 pmol/μl), 2 μl of 2.5 mM dNTP's, 2.5 μl 10× buffer, 2 μl of 25 mM MgCl₂ and 0.1 μl of Taq polymerase (0.5u). PCR cycles were carried out as described above. The PCR products were subsequently separated by agarose gel electrophoresis, and positive transformants selected on the basis of the correct band size. A positive control and a negative control were incorporated into the process to verify the results obtained.

Mutation Confirmation

Sequencing to confirm each mutation was outsourced. Forward and reverse gene-specific primers were designed from the known sequence of the gene for this purpose. These are shown in Table 5.

TABLE 5
Sequence-specific primers used for
confirming the presence of the various
mutations in the E. coli glnA gene

| | | |
|---|---|---|
| Primer | Mutation | Primer Sequence |

Forward Primers

| | | |
|---|---|---|
| GlnSeq 5 | S52A | 5′-GCTGAACACGTACTGACGATG-3′ |
| | S53A | (SEQ ID NO: 14) |
| | S52A | |
| | S53A | |
| GlnSeq 7 | E327V | 5′-GTCTGTCTGAGCAGGCGCTG-3′ |
| | Y397V | (SEQ ID NO: 15) |
| GlnSeq 9 | H210V | 5′-GCTATCGACGATATCGAAGG-3′ |
| | H211V | (SEQ ID NO: 16) |

Reverse Primers

| | | |
|---|---|---|
| GlnSeq 6 | S52A | 5′-GTGGGAACCGGAGATAGATGATC-3′ |
| | S53A | (SEQ ID NO: 17) |
| | S52A | |
| | S53A | |
| GlnSeq 8 | E327V | 5′-GCATAAAGTTTGGACGGCAA-3′ |
| | Y397V | (SEQ ID NO: 18) |
| GlnSeq 9 | H210V | 5′-GTTCTTGATACCATCAAGACCG-3′ |
| | H211V | (SEQ ID NO: 19) |

QuikChange™ Mutagenesis

DNA of the selected templates (pBluescript constructs) was isolated from E. coli XL1-Blue using the Qiagen MidiPrep Kit. The oligonucleotides designed to carry out the SDM using this system are listed in Table 6. As this is a PCR-based system, two oligonucleotides (sense and antisense) are required for each reaction.

TABLE 6
Mutations carried out on the E. coli
glnA gene using the QuikChange™ System.

| | | Restriction |
|---|---|---|
| Mutation | Oligonucleotide Sequence | Site |
| D50A | 5′-GAAGAAGGCAAAATGTTTGCAGGCTC- | ClaI |
| | ATCGATTGGCGGCTGG-3′ | |
| | (SEQ ID NO: 20) | |
| | 5′-GGAGCCGCCAATCGATGAGCCTGCAA- | |
| | ACATTTTGCCTTCTTC-3′ | |
| | (SEQ ID NO: 21) | |
| E129A | 5′-CCGTACTGTTCGGGCCCGCTCCTGAA- | ApaI |
| | TTCTTCCTGT-3′ | |
| | (SEQ ID NO: 22) | |
| | 5′-ACAGGAAGAATTCAGGAGCGGGCCCG- | |
| | ACAGTACGG-3′ | |
| | (SEQ ID NO: 23) | |
| E327V | 5′-GTCTGGTCCCGGGCTATGTAGCATCG- | ClaI |
| | ATAATGCTGG-3′ | |
| | (SEQ ID NO: 24) | |
| | 5′-CCAGCATTATCGATGCTACATAGCCC- | |
| | GGGACCAGAC-3′ | |
| | (SEQ ID NO: 25) | |
| E357A | 5′-GTCGTATCGCGGTACGTTTCCCGGAT- | PstI |
| | CCTGCAGCTAACCG-3′ | |
| | (SEQ ID NO: 26) | |
| | 5′-CGGTTAGCTGCAGGATCCGGGAAACG- | |
| | TACCGCGATACGAC-3′ | |
| | (SEQ ID NO: 27) | |

Mutations to change amino acid residues are shown in bold and restriction sites are underlined.

Following mutagenesis, single colonies were screened for the mutation of choice by isolating DNA from an overnight culture, and performing restriction analysis with the enzyme for the particular mutation being screened for. Positive mutants obtained with the QuikChange System were transformed into E. coli YMC11 for expression purposes. Sequencing to confirm each mutation was outsourced. D50A and E129A were sequenced using GlnSeq 5 and GlnSeq 6. E357A was sequenced using GlnSeq 9 and GlnSeq 10.

### Results

Cloning of the E. coli Glutamine Synthetase Gene

The wild type glnA gene of E. coli was amplified as a 2.1 kb fragment, encoding a protein of 471 amino acids in length. The 2124 bp PCR amplified glnA gene containing the NsiI flanking restriction sites was ligated into the PstI-digested SDM vector pAlter-1 at an insert:vector ratio of 3:1. DNA was isolated from a number of transformants and subjected to restriction analysis using BamHI and EcoRI. According to the known sequence of both the gene and the vector, restriction of a construct (with the glnA gene in the correct 5′ to 3′ orientation required) with BamHI, should produce fragments of 6012 bp and 1797 bp. A correct construct was identified in this way, and was named pGln12.

To construct the wild type template for the mutagenesis using the QuikChange System, the glnA gene was excised from pGln12 as a SacI-HindIII fragment, and ligated into similarly digested pBluescript II SK+ at an insert:vector ratio of 3:1. The ligation reaction was transformed into E. coli XL1-Blue and plated on LM agar supplemented with 100 μg/ml ampicillin, 80 μg/ml X-Gal and 1 mM IPTG. DNA was isolated from a number of white colonies and subjected to restriction analysis with SacI and HindIII and BamHI in order to identify a positive subclone. A correct construct was identified in this way and named pBSK-ECgln. Sequence analysis was performed on both clones to verify the integrity of the wild type gene before any SDM was carried out.

Site Directed Mutagenesis (SDM)

Altered Sites™ System

The site-directed mutagenesis was carried out as per the protocol described above. DNA isolated from the mutants was digested using the enzyme specific for the mutation, and size-fractionated to confirm the presence of the mutation. The presence of the mutation generally resulted in extra fragments as restriction sites were added. In all cases, the wild type construct (pGln12) was digested with the same enzyme as a comparative control. The mutations with their respective introduced restriction sites and expected fragment sizes are outlined in Table 7.

TABLE 7
List of mutations introduced into pGln12 with the Altered Sites ™
System, showing the expected restriction fragments.

Restriction
Fragments (bp)

| | Introduced | With | Without |
|---|---|---|---|
| Mutation | restriction site | Mutation | mutation |
| Y397V | SalI | 5918 | 7809 |
| | | 1891 | |
| S52A | ClaI | 6095 | 7809 |
| | | 1714 | |
| S53A | BamHI | 6012 | 6012 |
| | | 935 | 1797 |
| | | 862 | |
| S52A | PvuI | 5420 | 6457 |
| S53A | | 1352 | 1352 |
| | | 1037 | |
| H210V | NruI | 4439 | 4439 |
| | | 1478 | 1892 |
| | | 1189 | 1478 |
| | | 703 | |
| H211V | PvuII | 3047 | 3047 |
| | | 2254 | 2508 |
| | | 1493 | 2254 |
| | | 1015 | |
| E327V | ClaI | 6926 | 7809 |
| | | 883 | |

Agarose gel electrophoresis confirmed the presence of the mutations, as the expected DNA fragment sizes were obtained.

Once these nine single mutations had been confirmed, the double mutants were produced. In each case the Y397V mutation was added to each of the S52A, S53A, S52A S53A, H210V and H211V to produce S52A Y397V, S53A Y397V, S52A S53A Y397V, H210V Y397V and H211V Y397V, respectively. The mutations with their respective introduced restriction sites and the expected fragment sizes are outlined in Table 8.

TABLE 8
List of mutations introduced into Y397V with the Altered Sites
System, showing the expected restriction fragments.

| | |
|---|---|
| | Restriction |
| Introduced | Fragments (bp) |

| | restriction | With | Without |
|---|---|---|---|
| Mutation | site | Mutation | mutation |
| S52A Y397V | ClaI | 6095 | 7809 |
| | | 1714 | |
| | SalI | 5918 | 7809 |
| | | 1891 | |
| S53A Y397V | BamHI | 6012 | 6012 |
| | | 935 | 1797 |
| | | 862 | |
| | SalI | 5918 | 7809 |
| | | 1891 | |
| S52A S53A | PvuI | 5420 | 6457 |
| Y397V | | 1352 | 1352 |
| | | 1037 | |
| | SalI | 5918 | 7809 |
| | | 1891 | |
| H210V | NruI | 4439 | 4439 |
| Y397V | | 1478 | 1892 |
| | | 1189 | 1478 |
| | | 703 | |
| | SalI | 5918 | 7809 |
| | | 1891 | |
| H211V | PvuII | 3047 | 3047 |
| Y397V | | 2254 | 2508 |
| | | 1493 | 2254 |
| | | 1015 | |
| | SalI | 5918 | 7809 |
| | | 1891 | |

Agarose gel electrophoresis confirmed the presence of the mutations, as the expected DNA fragment sizes were obtained.

Mutant Gene Expression

Mutant genes (from pAlter) were subcloned into pBluescript II SK⁺ and transformed into E. coli YMC11, to facilitate protein purification and enzyme studies. The presence of the subcloned genes in the vector was confirmed by PCR screening. Transformant colonies containing the mutant glnA genes were detected as a 2170 bp band on an agarose gel. A negative control consisting of the vector transformed into E. coli YMC11 was included in the PCR screens, and appeared on the gel as a band the size of the vector multiple cloning site. A positive control of DNA of pBSK-ECgln, also in E. coli YMC11, was included. This appeared on the gel as a band the same size as any positive mutant subclones. The DNA from a single subclone of each mutant identified in this way was then digested with the restriction enzyme specific for the mutation to confirm the presence of the specific mutation.

TABLE 9
Outline of the expected restriction fragments of the mutant glnA
subclones.

Restriction
Fragments (bp)

| | | Fragment |
|---|---|---|
| Mutation | Enzyme | Sizes |
| pBSK-Y397V | SalI | 2907 |
| | | 1891 |
| | | 271 |
| pBSK-S52A | ClaI | 3771 |
| | | 1298 |
| pBSK-S52A | ClaI | 3771 |
| Y397V | | 1298 |
| | SalI | 2907 |
| | | 1891 |
| | | 271 |
| pBSK-S53A | BamHI | 3272 |
| | | 935 |
| | | 862 |
| pBSK-S53A | BamHI | 3272 |
| Y397V | | 935 |
| | | 862 |
| | SalI | 2907 |
| | | 1891 |
| | | 271 |
| pBSK-S52A | PvuI | 2542 |
| S53A | | 1482 |
| | | 1045 |
| pBSK-S52A | PvuI | 2542 |
| S53A Y397V | | 1482 |
| | | 1045 |
| | SalI | 2907 |
| | | 1891 |
| | | 271 |
| pBSK-H210V | NruI | 3177 |
| | | 1189 |
| | | 703 |
| pBSK-H210V | NruI | 3177 |
| Y397V | | 1189 |
| | | 703 |
| | SalI | 2907 |
| | | 1891 |
| | | 271 |
| pBSK-H211V | PvuII | 2513 |
| | | 1590 |
| | | 966 |
| pBSK-H211V | PvuII | 2513 |
| Y397V | | 1590 |
| | | 966 |
| | SalI | 2907 |
| | | 1891 |
| | | 271 |
| pBSK-E327V | ClaI | 4602 |
| | | 467 |

Agarose gel electrophoresis confirmed the presence of the mutations in the subclones, as the expected DNA fragment sizes.

QuikChange™ Mutagenesis

SDM using the QuikChange™ System was carried out as per the protocol set forth above. DNA was isolated from the possible mutants, digested using the enzyme specific for the mutation, and size-fractionated to confirm the presence of the mutation. A control consisting of the parent template was digested with the same enzyme as a comparative control. The mutations with their respective restriction sites and expected fragment sizes are outlined in Table 10. pBSK-ECgln was used as the template for the D50A, E129A and E357A mutations. The double mutants D50A Y397V, E129A Y397V, E327V Y397V and E357A Y397V were produced in pBSK-Y397V.

TABLE 10
List of mutations introduced into various templates with the
QuikChange System, showing the expected restriction fragments.

| | |
|---|---|
| | Restriction |
| Introduced | Fragments (bp) |

| | restriction | With | Without |
|---|---|---|---|
| Mutation | site | Mutation | Mutation |
| D50A | ClaI | 3771 | 5069 |
| | | 1298 | |
| E129A | ApaI | 3970 | 5069 |
| | | 1099 | |
| E357A | PstI | 5069 | Uncut |
| D50A | ClaI | 3771 | 5069 |
| Y397V | | 1298 | |
| | SalI | 2907 | 2907 |
| | | 1891 | 2162 |
| | | 271 | |
| E129A | ApaI | 3970 | 5069 |
| Y397V | | 1099 | |
| | SalI | 2907 | 2907 |
| | | 1891 | 2162 |
| | | 271 | |
| E327V | ClaI | 4602 | 5069 |
| Y397V | | 467 | |
| | SalI | 2907 | 2907 |
| | | 1891 | 2162 |
| | | 271 | |
| E357A | PstI | 5069 | Uncut |
| Y397V | SalI | 2907 | 2907 |
| | | 1891 | 2162 |
| | | 271 | |

Agarose gel electrophoresis confirmed the presence of the mutations, as the expected DNA fragment sizes were obtained.

### Conclusion

All the desired mutations were successfully produced using the selected site-directed mutagenesis systems. Silent mutations encoding restriction sites were included in the oligonucleotides designed for the mutagenesis. This allowed for primary screening without the need to sequence every potential mutant.

The following mutations were produced in the E. coli glutamine synthetase gene: Y397V, S52A, S52A Y397V, S53A, S53A Y397V, S52A S53A, S52A S53A Y397V, H210V, H210V Y397V, H211V, H211V Y397V, D50A, D50A Y397V, E129A, E129A Y397V, E327V, E327V Y397V, E357A and E357A Y397V.

### Example 3

### Complementation Studies and Purification of the Glutamine Synthetase Enzymes

Mutants were constructed to alter the residues thought to be involved in the proteolytic catalytic triads described above. These were the serine residues S52 and S53, the histidine residues H210 and H211, as well as D50, E129, E327 and E357. All the constructed mutants were tested by complementation of the glutamine synthetase auxotrophy in E. coli YMC11.

The various recombinant mutant glutamine synthetase enzymes were purified using a combination of streptomycin sulphate precipitation, pH changes and ammonium sulphate precipitation, until a pure enzyme preparation was achieved. An affinity column chromatography method was also developed and used to purify certain of the enzymes.

Two enzyme assays were utilised to assess glutamine synthetase activity. The first assay used, termed the γ-glutamyl transferase assay, is a variation of the reverse of the reaction that glutamine synthetase catalyses:

Glutamate+NH₄⁺+ATP→glutamine+ADP+Pᵢ+H₂O

In this reverse reaction, hydroxylamine and glutamine react to form γ-glutamylhydroxamate and free ammonia in the presence of ADP, arsenate and manganese or magnesium (Shapiro B M and Stadtman E R (1970) Methods in Enzymol. 17A: 910-922). This forms the basis of an assay for glutamine synthetase activity. At the correct pH, which is derived from determining the isoelectric point of the enzyme, the transferase activities of both the adenylylated and deadenylylated forms of glutamine synthetase are the same. The two forms can, however, be distinguished because at the isoelectric pointy, fully adenylylated glutamine synthetase is completely inhibited by 60 mM Mg²⁺, whereas the deadenylylated enzyme is unaffected.

In addition, the rate of conversion of ATP, glutamate and ammonia to glutamine and ADP was assessed using HPLC. The is termed the ‘forward’ or “biosynthetic” reaction and is assayed in two different assays; one which measures the ability of glutamine synthetase to convert glutamate to glutamine in the presence of ATP, and the second determines the conversion of ATP to ADP and AMP in the same assay mixture.

Methods and Materials

All chemicals were of analytical or molecular biology grade and were used without further purification. All chemicals were obtained from Merck or Sigma unless otherwise stated.

Mn(HCO₃)ATP was prepared in-house from Na₂ATP (obtained from Roche) which was dissolved in water to a concentration of approximately 80 mM. The Na⁺ ions were then removed from the ATP by passing the solution over a Dowex 50 WX2 strong cation exchange resin. All samples containing the acid-ATP were pooled and reacted with an equivalent molar concentration of Mn(HCO₃). The solution was stirred until all the MnCO₃ was dissolved. The pH of the Mn(HCO₃)-ATP solution was then adjusted to pH 7.0 with NaHCO₃.

SDS Polyacrylamide gel electrophoresis (SDS-PAGE) was carried out according to standard protocols (Laemmli U K (1970) Nature 227: 680-685). Acrylamide was purchased from Sigma as a 40% Acrylamide:bis-acrylamide:mixture (19:1 ratio). The broad range protein molecular weight marker from Fermentas was used for all PAGE gels.

Complementation of the Glutamine Auxotrophy in E. coli YMC11

Single colonies of each of the mutant recombinant glutamine synthetase constructs in pBluescript II SK⁺ in the E. coli auxotrophic strain YMC11 were plated on M9 Minimal Medium (Sambrook, J., Fritsch, E. F. and T. Maniatis (1989) In: Molecular Cloning: A Laboratory Manual., 2^{nd} ed. Cold Spring Harbor Laboratory Press.), to assess their ability to complement the auxotrophy of E. coli YMC11. Glutamine, at a concentration of 250 mg/l, was added to one plate, and a duplicate plate contained no glutamine. Ampicillin was added to both plates at a concentration of 100 μg/ml. E. coli YMC11 transformed with non-recombinant pBluescript II SK⁺ was used as a negative control. The wild type recombinant construct, pBSK-ECgln was used as the positive control. The plates were incubated at 37° C. and observed over a 48 hour period for the presence or absence of growth.

Purification of the E. coli Glutamine Synthetase

All recombinant constructs used for the isolation of GS were cultured in 2 L of a modified M9 medium (6 g/l Na₂HPO₄, 3 g/l KH₂PO₄, 0.5 g/l NaCl) supplemented with 70 mM L-glutamate, 5 mM L-glutamine and 100 μg/ml ampicillin. All cultures were incubated at 37° C. for 48 hours with shaking at 220 rpm. Cells were harvested from the culture medium by centrifugation at 10 000 rpm at 4° C. The biomass was then either used fresh or stored at −20° C. until used.

In addition, the wild type glutamine synthetase (from pBSK-ECgln) was purified in both the adenylylated and deadenylylated forms, from biomass obtained from continuous culture being carried out as part of another investigation. Adenylylated enzyme was produced under conditions of nitrogen limitation and carbon excess, while deadenylylated enzyme was produced under conditions of nitrogen excess and carbon limitation (Senior P J (1975) Journal of Bacteriology 123: 407-418). The cells obtained were harvested by centrifugation at 10 000 rpm for 10 minutes at 4° C., and stored at −20° C. until required. The method used for the purification of glutamine synthetase was developed from the method of Shapiro B M and Stadtman E R (1970) Methods Enzymol. 17A: 910-922.

The biomass was resuspended in 10 mls of Resuspending Buffer A or RBA (10 mM Imidazole-HCl, 2 mM β-mercaptoethanol, 10 mM MnCl₂.4H₂0; pH 7.0). The cells were sonicated for 10 minutes on a 50% duty cycle. This sonicated solution was centrifuged for 10 minutes at 10 000 rpm, and the supernatant was retained. Streptomycin sulphate was added (10% of a 10% w/v), and the suspension was stirred at 4° C. for 10 minutes. Centrifugation was then carried out at 10 000 rpm for 10 minutes and the supernatent was retained. The pH of the supernatant was adjusted to 5.15 with sulphuric acid. This mixture was stirred at 4° C. for 15 minutes, and then centrifuged at 13 000 rpm for 10 minutes. Again, the supernatant was retained. Saturated ammonium sulphate (30% of volume) was added and the pH was adjusted to 4.6 with sulphuric acid. The suspension was stirred at 4° C. for 15 minutes, and then centrifuged at 13 000 rpm for 10 minutes. The precipitate obtained was resuspended in 2-5 mls of RBA and the pH adjusted to 5.7 with sulphuric acid. This suspension was stirred overnight at 4° C. to allow the glutamine synthetase to resuspend, and then centrifuged at 13 000 rpm for 10 minutes. The supernatant was retained and the pH of the suspensions was adjusted to 7.0.

Further purification of the wild type and the Y397V enzymes was achieved through the use of affinity chromatography using an AKTAExplorer (Amersham Biosciences). Separation was achieved with 5′AMP Sepharose resin with an HR10/10 column which has a length of 10 cm and an internal diameter of 10 mm. The glutamine synthetase enzyme preparation (approximately 2 mls) was loaded onto the prepared column which was equilibrated with 10 mM Imidazole (pH 7.0), 150 mM NaCl and 10 mM MnCl₂.4H₂O. The bound glutamine synthetase was eluted off the column with 2.5 mM ADP across a 40 ml linear gradient 150 to 500 mM NaCl, and 1 ml fractions were collected. The fractions containing pure glutamine synthetase were then pooled and dialysed overnight against RBA.

An aliquot of each protein suspension was electrophoresed on a 7.5% SDS PAGE gel according to standard protocols. Protein concentration was determined using the Lowry protein determination method.

MALDI-TOF Analysis of Purified Proteins

Each mutant protein was analysed using MALDI-TOF mass spectroscopy. The desired protein band was excised from a 7.5% SDS PAGE gel (multiple lanes of the same protein were run to obtain sufficient protein). The desired bands were excised from the gel and placed into an Eppendorf tube. Sixty μl of a solution of 75 mM NH₄(HCO)₃ in 40% ethanol was added to the gel plug and vortexed for 30 minutes. The liquid was removed and discarded. This destaining procedure was repeated until no Coomassie blue remained in the gel plug. The gel plug was then covered with acetonitrile, and incubated at room temperature for 15 mins, following which all liquid was removed from the tube leaving the gel plug. Sufficient volume of a solution of 5 mM dithiothreitol in 50 mM ammonium bicarbonate was then added to the tube to cover the gel plug and this was incubated at 60° C. for 30 mins. The suspension was then centrifuged at 13 000 rpm for 5 mins in an Eppendorf microfuge, and the supernatant was removed and discarded.

Ten μl of a solution of 55 mM Iodoacetamide in 50 mM ammonium bicarbonate was added to the gel plug and incubated at room temperature for 60 mins in the dark. Following incubation, the suspension was centrifuged at 13 000 rpm for 5 mins and the supernatant was removed and discarded. The gel plug was covered with 60 μl of acetonitrile, and this suspension was allowed to stand for 10 mins. Following 5 mins of centrifugation at 13 000 rpm the supernatant was discarded, and the gel plug was dried in a Speedvac for 10 mins.

Freshly prepared bovine trypsin (5-20 μl) was then added at a concentration of 0.01 μg/μl in 50 mM NH₄HCO₃. The amount added is dependent on the amount of gel in the Eppendorf tube. This is incubated on ice for 60 mins. After brief centrifugation, the supernatant was removed and 10 μl of 50 mM NH₄HCO₃ was added to the gel plug. This was incubated at 37° C. overnight.

The sample was then sonicated for 10 min in a sonicating bath and the supernatant was recovered after centrifugation in an Eppendorf microfuge for 5 mins. Ten μl of a Tri-fluoroacetic acid (TFA)/acetonitrile solution (50% of 2% TFA and 50% acetonitrile) was added and the sample was sonicated again for 10 mins. After centrifugation, the supernatant was recovered. Acetonitrile (10 μl) was added and the sample vortexed for 10 mins. The sample was centrifuged at 13 000 rpm for 5 mins and the supernatent retained. Equal volumes of the prepared protein sample and a 10 mg/ml solution of alpha-cyano hydroxyl cinnamic acid in 01% TFA in acetonitrile were then combined. This was mixed vigorously, and 1 μl of the solution applied to the sample plate. The proteins were then analysed by MALDI TOF MS.

Determination of Glutamine Synthetase Activity Using the Glutamyl Transferase Assay

The γ-glutamyl transferase assay was used to measure the total amount of glutamine synthetase present, since both the adenylylated and deadenylylated forms of the enzyme are active in this assay in the presence of Mn²⁺. When the same assay is supplemented with 60 mM Mg²⁺ the activity of only the deadenylylated enzyme is determined. The activities of the two forms of the enzyme can therefore be differentiated on the basis of the difference in activity in the presence of Mn²⁺ or Mg²⁺. The assay mixture was adapted from Shapiro B. M. and Stadtman E. R. (1970) Annual Reviews in Microbiology 24:501-524.

Glutamine synthetase activity is measured in two different assay mixtures: one containing only Mn and a second containing both Mn and Mg. All reagents were prepared in Imidazole Buffer (pH 7.0). Both assays were run in a total volume of 600 μl. The Mn assay was set up as shown in Table 11 below.

TABLE 11
Assay Mixture for the Mn-based glutamyl transferase assay

| | | Final Concentration |
|---|---|---|
| Component | Concentration | in Assay |
| L-Glutamine | 112.5 mM | 15 mM |
| NaADP | 4 mM | 0.4 mM |
| Sodium Arsenate | 180 mM | 30 mM |
| MnCl₂4H₂O | 4.5 mM | 0.3 mM |
| Hydroxylamine | 900 mM | 60 mM |

The combination (n and Mg) assay was set up as shown in Table 12.

TABLE 12
Assay Mixture for the Mn and Mg-based glutamyl transferase
assay

| | | Final Concentration |
|---|---|---|
| Component | Concentration | in Assay |
| L-Glutamine | 112.5 mM | 15 mM |
| NaADP | 4 mM | 0.4 mM |
| Sodium Arsenate | 180 mM | 30 mM |
| MnCl₂•4H₂O | 4.5 mM | 0.3 mM |
| MgCl₂•7H₂O | 900 mM | 60 mM |
| Hydroxylamine | 900 mM | 60 mM |

A blank reaction was prepared in the same manner as the Mn reaction, but replacing the ADP and arsenate solutions with water.

The assay mix was equilibrated for 5 mins at 37° C., and then initiated by the addition of 50 μl of enzyme preparation. The reaction was allowed to proceed for 30 mins, and then terminated by the addition of 900 μl of Stop Mix (1M FeCl3.6H₂O, 0.2M Trichloroacetic acid and 7.1% v/v HCl). The samples were then centrifuged at 13000 rpm for 2 mins in an Eppendorf microfuge to remove any precipitate that may have formed, and the absorbance measured at 540 nm. All absorbance readings were entered into a spreadsheet, and the results presented as specific activity in terms of μmoles enzyme/min/mg protein. The degree of adenylylation was calculated from the ratio of the deadenylylated γ-glutamyl transferase activity to the total γ-glutamyl transferase activity (Mn²⁺ reaction), taking the number of subunits into account.

Determination of Glutamine Synthetase Activity by HPLC

This assay was set up to measure glutamine synthetase activity according to the forward reaction, and measures the amount of glutamine formed from 4 millimoles of L-glutamate in the presence of MnCO₃-ATP (the basis of one reaction) and MgATP (the basis of a second reaction). In addition, the amount of ADP formed is also measured in a separate assay, using the same reagents. The assay set-up is shown in Table 13.

TABLE 13
Assay Components for the HPLC assay to determine the rate of
conversion of glutamate and ATP to glutamine and ADP.

| Mn Assay | | Mg Assay | |
|---|---|---|---|
| | Concentration | | Concentration |
| Component | in Assay | Component | in Assay |
| MnHCO₃- | 4 mM | Na-ATP | 4 mM |
| ATP | | | |
| L-Glutamate | 4 mM | L-Glutamate | 4 mM |
| NH₄HCO₃ | 4 mM | (NH₄)₂HPO₄ | 2 mM |
| | | MgCl₂•6H₂O | 4 mM |

All enzyme preparations were adjusted to the same concentration of protein, and added to the assay mixture in a volume of 50 μl. The addition of the enzyme started the reaction, which was then allowed to proceed for 1 hour. The reaction was stopped by the addition of 6 μl of a 50% solution of trichloroacetic acid. Each assay was then aliquoted into 4 HPLC vials (150 μl per vial), two of which were assayed for glutamate and glutamine using a Phenomenex Luna 5 μC18 Column on an Agilent 100 HPLC instrument. The ATP/ADP assays were run on a Chromolith Performance RP-18c column also using an Agilent 100 HPLC instrument.

As it is believed that two putative catalytic triads, similar to those in the serine proteases, are involved in the reaction mechanism of glutamine synthetase, certain of the mutants were also assayed by HPLC in the presence of the serine protease inhibitors, AEBSF and PMSF, in order to assess the effect that these inhibitors had on the enzyme. The enzymes assessed were the adenylylated WT enzyme, the deadenylylated WT enzyme, and the mutant enzymes S52A, S52A Y397V, S53A, S53A Y397V, S52A S53A and S52A S53A Y397V. The enzymes were pre-incubated for 60 minutes in the presence of 1 mM PMSF or 1 mM AEBSF. A control reaction was set up for each enzyme, which contained no inhibitor. Each enzyme was then added to assays set up as described above. The assay was allowed to proceed for 60 minutes, then stopped by the addition of 6 ml of 50% TCA, after which they were analysed by HPLC. All assays were run in triplicate.

### Results

Complementation Studies

The mutant glutamine synthetase gene constructs were grown on M9 minimal medium agar in the presence and absence of glutamine, and assessed for their ability to complement the glnA mutation of E. coli YMC11. After 48 hours of incubation at 37° C., it was observed that while the negative control (pBluescript II SK⁺ in YMC11) was unable to grow in the absence of glutamine, the positive control (pBSK-ECgln) was capable of complementing the auxotrophy in YMC11. A problem that was often experienced was that 250 mg/l of glutamine appears to be insufficient to support the growth of the YMC11 strain, and therefore the vector alone in YMC11 struggled to grow. The presence of a functional multicopy glutamine synthetase gene, as in the positive control, ECgln, appears to overcome this problem. pBSK-Y397V, the recombinant deadenylylated construct, was also capable of complementing the auxotrophy of YMC11. Both the WT enzyme and the Y397V enzyme are therefore functional.

The S52A and S53A mutants as well as the double mutants, S52A Y397V and S53A Y397V, were all capable of complementing the auxotrophy of E. coli YMC 11, thus indicating functionality. The same was found to be true for the double serine mutant, S52A S53A, and its counterpart, S52A S53A Y397V. The two histidine mutants, H210V and H211 V, grew well on the minimal plate supplemented with glutamine, but grew very badly on the plate containing no glutamine, thus indicating enzymes with little functionality. The double histidine mutants, H210V Y397V and H211V Y397V, on the other hand, grew very badly even on the plate supplemented with glutamine, and not at all on the plates containing no glutamine. It was therefore assumed that these two enzymes had virtually no functionality and that these mutations created auxotrophy, with the result that they have an absolute requirement for glutamine.

Mutants D50A and D50A Y397V exhibited good growth on both plates, and are both functional. Both E129A and E357A, however, were not capable of complementing the auxotrophy of YMC11, and both exhibited very poor growth even on the plate supplemented with glutamine. The same applied to the double mutants, E129A Y397V and E357A Y397V. E327V and E327V Y397V exhibited good growth on both plates, and both have functional glutamine synthetase enzymes.

Purification of the E. coli GS

For the purification of the mutant proteins, each mutant was grown in a modified M9 minimal medium supplemented with glutamate as the sole nitrogen source. A small amount of glutamine was added to facilitate cell growth, as the YMC11 strain is a glutamine auxotroph.

All cultures were grown for 48 hours at 37° C., and then checked for contamination. Recombinant plasmid integrity was checked by isolating the plasmid DNA, subjecting it to restriction analysis, followed by agarose gel electrophoresis.

It was found that some of the double mutants, namely H210V Y397V, H211V Y397V and E357A Y397V required the addition of 110 mM glutamine to the minimal medium in order to get sufficient biomass to enable enzyme purification, as the growth was so poor. Glutamine synthetase enzyme was successfully purified to at least 90% homogeneity from all the mutants. In all instances, the presence of purified glutamine synthetase was demonstrated as the presence of a single band at approximately 55 kDa.

Verification of Purified Glutamine Synthetase

All the purified mutant glutamine synthetase enzymes were subjected to MALDI TOF analysis to verify that the enzymes were glutamine synthetase. Each enzyme was run on a 7.5% SDS-PAGE gel and the bands expected to be glutamine synthetase were cut out and the protein was purified as described. Purified protein samples were pipetted onto a sample plate, and subjected to analysis by MALDI TOF mass spectroscopy. The proteins were then identified by using proteomics and sequence analysis tools and entering the values obtained into the database of the ExPASy Proteomics server of the Swiss Institute of Bioinformatics. All the isolated proteins were identified as glutamine synthetase in this manner.

### Glutamyl Transferase Assays

Each mutant was assessed for activity using the γ-glutamyl transferase or “reverse” reaction. This determines the presence of a ferric-hydroxamate complex in a colourimetric assay in which the absorbance is read at 540 nm n. The assay determines total enzyme activity in a reaction containing Mn²⁺. In addition, the degree of adenylylation of the glutamine synthetase enzyme being screened is also determined in a reaction containing both Mn²⁺ and Mg²⁺, as the adenylylated form of the enzyme is inhibited in the presence of Mg²⁺ (Shapiro B M and Stadtman E R (1970) Annual Reviews in Microbiology 24: 501-524; Bender R A, K A Janssen, A D Resnick, M Blumberg, F Foor and B Magasanik (1977) Journal of Bacteriology 129: 1001-1009). In this way, the degree of adenylylation of the glutamine synthetase enzyme can be determined. The results of the assays are shown in Table 14, with the enzyme activities shown in μmoles per minute per mg of protein.

TABLE 14
Glutamyl transferase assay results of the E. coli WT and
constructed mutants. The WT enzyme refers to the strain grown in the
modified M9 medium, and the WT (AD) and WT (DD) refers to the
adenylylated and deadenylylated enzymes, respectively, produced in
continuous culture. The values presented represent the average of
at least three assays where the variation in activity was less than 10%.

| | | Activity of | |
|---|---|---|---|
| | | deadenylylated | |
| | Total Enzyme | Enzyme: | |
| | Activity: | 4.5 mM MnCl₂ + | |
| | 4.5 mM MnCl₂ | 60 mM | |
| | (μmoles/min/mg | MgCl₂ (μmoles/min/ | Percentage of |
| | protein) | mg protein) | Adenylylaton |
| WT | 91.5 | 20.2 | 78 |
| WT (AD) | 84.9 | 21.1 | 75 |
| WT (DD) | 55.8 | 56.1 | 0 |
| Y397V | 64.5 | 5.1 | 92 |
| Catalytic Triad | | | |
| S52A | 35.5 | 8.9 | 75 |
| S52A Y397V | 78.6 | 14.0 | 82 |
| S53A | 47.8 | 19.1 | 60 |
| S53A Y397V | 86.2 | 30.5 | 65 |
| S52A S53A | 7.2 | 2.8 | 61 |
| S52A S53A | 29.9 | 7.5 | 75 |
| Y397V | | | |
| H210V | 2.8 | 0.0 | 100 |
| H210V Y397V | 0.0 | 0.0 | — |
| H211V | 0.0 | 0.0 | — |
| H211V Y397V | 0.0 | 0.0 | — |
| D50A | 1.28 | 0.60 | 53 |
| D50A Y397V | 1.78 | 0.20 | 89 |
| E129A | 2.38 | 0.60 | 75 |
| E129A Y397V | 12.06 | 2.05 | 83 |
| E327V | 0.7 | 0.0 | 100 |
| E327V Y397V | 1.73 | 0.32 | 82 |
| E357A | 3.3 | 3.5 | 0 |
| E357A Y397V | 0.03 | 0.05 | 0 |

The WT enzyme grown in the same way as the mutants in a modified-M9 medium (WT in the table), is largely in the adenylylated form as most of the activity is inhibited approximately 4-fold from 91 to 20 μmoles/min/mg protein, in the presence of Mg²⁺, giving a percentage of adenylylation of 78%. The same percentage of adenylylation is seen in the WT strain grown under continuous culture conditions of N excess and C limitation, where the glutamine synthetase would be predominantly adenylylated (WT (AD) in the Table). The WT enzyme, grown in N limitation and C excess continuous culture, should be in the deadenylylated form (WT (DD) in the Table) and this is reflected in the assay results. The specific activities for this particular strain are almost identical in the assay run in the presence of Mn²⁺ only (55.8), as in the presence of Mn²⁺ and Mg²⁺ (56.1), giving, as would be expected, a percentage of adenylylation of 0.

The mutant enzyme, Y397V, was expected to be in the deadenylylated form, as the Tyr397 has been substituted by a valine residue. The mutant showed a very high total activity of 64.5 μmoles/min/mg protein, but does not appear to be deadenylylated; the percentage adenylylation appeared to in fact be higher, as the activity in the presence of Mn²⁺ and Mg²⁺ (5.1) is much lower than that detected in the presence of Mn²⁺ only (64.5), resulting in a percentage of adenylylation of 92%. This could be due to the fact that the adenyltransferase is incapable of adenylylating or deadenylylating the valine residue, resulting in a glutamine synthetase enzyme that is improperly folded. It is postulated that, the adenylylation and deadenylylation events carried out by the adenyltransferase entail the specific folding of the glutamine synthetase loop, by the adenyltransferase, into either one of two conformations required for each state of the enzyme, with the steric effect of the adenine groups facilitating the folding of the enzyme into the correct conformation. The Y397V mutant enzyme may, therefore have a loop conformation similar to the adenylylated form of the enzyme, as the adenyltransferase would not have folded the Y397 loop into the “correct” conformation required for deadenylylation. The adenylylated form of this enzyme, therefore, may tend towards being the default structure.

The two serine residues, 52 and 53, were altered singly in two mutant strains and both of these mutations resulted in a lowering of the total γ-glutamyl transferase activity, to 35.5 and 47.8 μmoles/min/mg of protein, respectively, indicating that these residues are essential for catalytic activity. This was reinforced by the mutagenesis that altered both serine residues to alanine, resulting in a double mutant, S52A S53A. In this mutant, the γ-glutamyl transferase activity was severely reduced to 7.2 μmoles/min/mg protein. Altering the serine residues each separately, together with the Y397V mutation, to produce S52A Y397V and S53A Y397V, resulted in an increase in the Mn²⁺ activity over that seen in the two individual serine mutants, to levels comparable with the WT enzyme (78.6 and 86.2 μmoles/min/mg protein). The percentage of adenylylation in S52A and S52A Y397V is similar to that obtained for the WT (all in the region of 75-80%). Altering S53 results in a lowering of the percentage of adenylylation to 60-65% in both mutants (S53A and S53A Y397V), possibly indicating that S53 is an important residue for deadenylylation. Altering both the serine residues simultaneously, resulting in S52A S53A, resulting in a large reduction in γ-glutamyl transferase activity to 7.2 μmoles/minmg protein. When the triple mutant, S52A S53A Y397V was assayed, some γ-glutamyl transferase activity was restored (29.9 μmoles/min/mg protein), but this activity was still less than that obtained in the WT strains. This would seem to indicate that, as suspected, these serine residues play an important role in the glutamine synthetase enzyme.

If these serine residues played a catalytic role similar to the serine residues making up the catalytic triad in serine protease, one would expect that the double serine mutation (S52A S53A) would create a non-functional enzyme, unless another reaction mechanism is available to this enzyme or the role they play in the “reverse reaction” or γ-glutamyl transferase reaction may be purely structural and not catalytic i.e. the glutamyl transferase reaction does not go via an acyl intermediate at the seine residues.

The two histidine residues identified as potentially forming part of a catalytic triad, proved to be crucial to the catalytic activity of the enzyme. H210V exhibited a very small amount of Mn²⁺-dependent γ-glutamyl transferase activity (2.8 μmoles/min/mg protein), which decreased to nothing in the double mutant H210V Y397V. Neither mutant of H211V exhibited any activity at all. H210V was fully adenylylated (percentage of adenylylation of 100%). When the ability to adenylylate or deadenylylate this enzyme was removed in the double mutant H210V Y397V, no activity was obtained.

The four residues identified as potentially being the acid residue of the catalytic triad, i.e., D50A, E129A, E328V and E357A, all showed varying amounts of activity. D50A and D50A Y397V show a small amount of total γ-glutamyl transferase activity (1.28 and 1.78 μmoles/min/mg protein, respectively), but show different degrees of adenylylation. E129A shows a small amount of activity (2.38 μmoles/min/mg protein), which increases to 12.06 μoles/min/mg protein in the double mutant E129A Y397V. None of the mutants E328V, E328V Y397V, E357A and E357A Y397V exhibit much γ-glutamyl transferase activity, the highest amount being 3.3 μmoles/min/mg protein shown by E357A. Both E357A mutants showed 0% adenylylation, although this is off of a very low activity base. As observed in the literature, these four residues are important for catalytic activity as the introduction of the mutations resulted in a reduction in activity in all instances (Liaw S-H, C Pan and D Eisenberg (1993a) Proceedings of the National Academy of Sciences 90: 4996-5000; Liaw S-H and D Eisenberg (1994a) Biochemistry 33: 675-681; Alibhai M and J J Villafranca (1994) Biochemistry 33:682-686.)

HPLC Assays

In addition to the γ-glutamyl transferase assays, assays were also developed to measure the rate of conversion of ATP and glutamate to ADP and glutamine by HPLC. These were set up as single assays but analysed separately for glutamate and glutamine and ATP and ADP. The conversion rates for glutamate to glutamine, in both the presence of Mn²⁺ and Mg²⁺ by glutamine synthetase, is referred to as the glutamine-based glutamine-based specific activity and is presented in μmoles/min/mg protein. The conversion rates for ATP to ADP, in both the presence of Mn²⁺ and Mg²⁺ by glutamine synthetase, is referred to as the ADP-based specific activity and is presented in μmoles/min/mg protein. The percentage of conversion was also calculated by dividing the glutamine-based specific activity by the ADP-based specific activity, and this value is interpreted as the ability of the enzyme to complete both steps of the reaction, i.e., glutamate to glutamine and ATP to ADP, at an equivalent level of efficiency.

The WT enzyme grown in the minimal medium shows similar glutamine- and ADP-based specific activities (1.3058 and 1.3651 μmoles/min/mg protein, respectively) and a conversion rate of 100%.

The WT enzyme grown under conditions of nitrogen limitation and carbon excess to adenylylated shows very similar levels of activity in both the Mn and Mg assays and has a conversion rate of 100%. The WT enzyme grown under conditions of nitrogen excess and carbon limitation to be deadenylylated was more active in the Mg assays (0.4412 mmoles/min/mg protein in the glutamine assay and 0.4537 μmoles/min/mg protein in the ADP assay) than in the Mn assays (0.1425 μmoles/min/mg protein in the glutamine assay and 0.1409 μmoles/min/mg protein in the ADP assay). Both the Mn and Mg assays showed a percentage of conversion close to 100%. As the deadenylylated enzyme should exhibit more Mg activity than adenylylated enzyme, this result was to be expected.

The Y397V enzyme is more active in the Mn²⁺ assays than in the Mg²⁺ assays, with conversion rates of only 50%. This low conversion efficiency is possibly due to the incorrect folding of the Tyr397 flexible loop producing an active site containing unbound water. This water can then act as the nucleophile, reacting with the highly unstable glutamyl phosphate intermediate converting it back to glutamate and PO₄, creating the inefficiency. In addition, as postulated that the adenylylation of the enzyme by the adenyltransferase entails the adenylylation of the Tyr397 flexible loop, and the specific conformation of this loop is induced by the steric effects of the adenine groups, facilitating the folding of the enzyme into the correct conformation.

S52A and S52A Y397V are both very active and show more glutamine synthetase activity than the WT enzymes screened. S52A produced similar activity levels in both the glutamine- and ADP-based Mn and Mg assays (in the region of 2.0 to 2.8 μmoles/min/mg protein), with a percentage of conversion of 91% for the Mn assays and 85% for the Mg assays. S52A Y397V, on the other hand was more active than S52A producing a glutamine-based Mn specific activity of 5.366 μmoles/min/mg protein and an ADP-based Mn specific activity of 6.2410 μmoles/min/mg protein. This was equivalent to a conversion rate of 91%. The Mg assays gave a glutamine-based Mg specific activity of 5.3063 μmoles/min/mg protein and an ADP-based Mg specific activity of 7.6781 μmoles/min/mg protein, representing a percentage conversion of 69%.

S53A produced activity levels slightly higher than those produced in the WT strains—5.80 μmoles/min/mg protein for the Mn²⁺ glutamine-based assay, 6.36 μmoles/min/mg protein for the Mn²⁺ ADP-based assay, 4.88 μmoles/min/mg protein for the Mg²⁺ glutamine-based assay and 6.96 μmoles/min/mg protein for the Mg²⁺ ADP-based assay. These represented conversion rates of 91% and 70%, respectively. The activity levels found for S53A Y397V were significantly higher than those found in the WT strains. The Mn²⁺ activities increased to 15.45 for the glutamine-based assay and 21.32 μmoles/min/mg protein for the ADP-based assay. The Mg²⁺ activities were 10.66 μmoles/min/mg protein for the glutamine-based assay and 15.42 μmoles/min/mg protein for the ADP-based assay.

S52A S53A was not as active as the two S53A mutants. It produced Mn activities of 2.2873 μmoles/min/mg protein for the glutamine assay and 2.5904 μmoles/min/mg protein for the ADP assay, with a percentage of conversion of 88%. The enzyme did, however exhibit the ability to convert ATP right through to AMP in the presence of Mn, with a specific activity of 0.3858 μmoles/min/mg protein being obtained for the conversion of ADP to AMP. S52A S53A did exhibit much lower Mg activity levels—0.9094 μmoles/min/mg protein for the glutamine assay and 1.2676 μmoles/min/mg protein for the ADP assay. This lower activity may be occurring in the active site attributed to the adenylylated form of the enzyme. The enzymes capable of converting ATP to AMP were able to synthesize glutamine from ADP (data not shown).

When the S52A S53A mutation was combined with the Y397V mutation, the Mn activity levels increased compared to the S52A S53A mutant (to 3.095 μmoles/min/mg protein for the glutamine assay and 4.5381 μmoles/min/mg protein for the ADP assay). The Mg glutamine- and ADP-based specific activities for this triple mutant, however, decreased quite significantly to 0.1075 and 0.2986 μmoles/min/mg protein. Again, this enzyme exhibited the ability to produce AMP in the assay, showing a specific activity of 0.3583 μmoles/min/mg protein. These results show that the two serine residues are very important for the catalytic activity of the enzyme. The double mutation, S52A S53A, did not create auxotrophy in the strain, as would be expected. This however be may be explained by the fact that these enzymes are capable of producing glutamine from ADP as well as ATP. As the conversion efficiency of these enzymes is severely compromised it would appear the reaction may be occurring directly from the γ-glutamyl phosphate and not the acyl enzyme intermediate, as the phosphoric acid anhydride would be unstable in the presence of H₂O allowing the hydrolysis of the γ-glutamyl phosphate and creating the conversion inefficiency.

The two histidine residues believed to form part of the catalytic triad, in general, showed very low levels of activity. H210V was the only mutant to show activity levels comparable to the WT enzymes, and then only in the Mn assay (0.1525 μmoles/min/mg protein for the glutamine-based assay and 0.1355 μmoles/min/mg protein for the ADP-based assay). The levels of activity produced in the Mg assay were very low and were considered to be equivalent to no activity (0.0348 μmoles/min/mg protein for the glutamine-based assay and 0.0173 μmoles/min/mg protein for the ADP-based assay). The double mutant H210V Y397V showed very little activity in any assay—0.0074 μmoles/min/mg protein in the Mn glutamine-based assay, 0.0025 μmoles/min/mg protein in the Mn ADP-based assay, 0.0036 μmoles/min/mg protein in the Mg glutamine-based assay and 0.0073 μmoles/min/mg protein in the Mg ADP-based assay. Neither H211V or H211V Y397V exhibited much activity in any assay, indicating their importance in the active site of glutamine synthetase. All four mutants had specific activities in all the assays of less than 0.1 μmoles/min/mg protein. It was interesting to note, however, that all four of these mutants could convert ATP all the way to AMP, although a lower level of AMP was produced than for the double serine mutants.

Of the residues identified as the potential acid residue in the putative catalytic triad, viz D50A E129A, E327V and E357A, all showed reduced levels of activity, and varying rates of conversion. D50A showed reduced activity compared to the WT clones. This mutation, when combined with the Y397V mutation, resulted in an increase in activity in all the assays, compared to D50A. The Mn²⁺ glutamine-based assay specific activity and the Mn²⁺ ADP-based assay specific activity increased from 0.62 μmoles/min/mg protein and 0.92 μmoles/min/mg protein, respectively, in D50A to 2.82 μmoles/min/mg protein and 4.90 μmoles/min/mg protein, respectively, in D50A Y397V. The Mg²⁺ activities showed a similar trend, increasing from 0.29 μmoles/min/mg protein and 0.85 μmoles/min/mg protein, respectively, in D50A to 1.98 μmoles/min/mg protein and 4.19 μmoles/min/mg protein, respectively, in D50A Y397V. The conversion rates in both mutants were low, indicating that the enzyme was not fully functional. E129A exhibited no activity in any assay. Some activity became detectable in the double mutant, E129A Y397V. E327V showed low specific activities in the Mn²⁺ assay (0.32 μmoles/min/mg protein in the glutamine-based assay and 0.42 μmoles/min/mg protein in the ADP-based assay, but these levels dropped in the Mg²⁺ assay. E327V Y397V, on the other hand, had similar Mn²⁺ glutamine-based and ADP-based specific activities to those obtained in E327V, but the Mg²⁺ activities were significantly higher than the Mg²⁺ activities of E327V. This could be an indication that this residue is important in the adenylylated form of the enzyme. E357A produced specific activities in the Mn²⁺ assay of 0.36 μmoles/min/mg protein glutamine-based activity and 0.21 μmoles/min/mg protein ADP-based activity. No activity was detected in either Mg²⁺ assay. When E357A was combined with Y397V, the double mutant showed lower Mn²⁺ activities than E357A, but Mg²⁺ activity was restored (0.04 μmoles/min/mg protein glutamine-based activity and 0.01 μmoles/min/mg protein ADP-based activity).

TABLE 15
HPLC Assay results showing the rate of conversion of ATP and
glutamate to glutamine, ADP and Pi as determined using HPLC. The WT
enzyme refers to the strain grown in the modified M9 medium, and the WT
(AD) and WT (DD) refer to the adenylylated and deadenylylated enzymes
produced in continuous culture. The values presented represent the
average of three different assays, in which the difference of the values was less than 5%.

| | | ADP- | | | ADP- | | AMP- |
|---|---|---|---|---|---|---|---|
| | Glutamine- | Based | | Glutamine- | Based | | Based |
| | Based | Specific | | Based | Specific | | Specific |
| | Specific | activity | | Specific | activity | | activity: |
| | activity | Mn | | activity | Mg | | Mn²⁺ |
| | Mn Assay | Assay | | Mg Assay | Assay | | Assay |
| | (μmoles/min/ | (μmoles/ | Percentage | (μmoles/min/ | (μmoles/ | Percentage | (μmoles/ |
| | mg | min/mg | Conversion | mg | min/mg | Conversion | min/mg |
| | protein) | protein) | Mn Assay | protein) | protein) | Mg Assay | protein) |
| WT | 4.31 | 4.37 | 98 | 4.26 | 4.21 | 100 | — |
| WT | 3.76 | 3.65 | 100 | 3.02 | 3.12 | 97 | — |
| (AD) | | | | | | | |
| WT | 4.98 | 4.62 | 100 | 14.54 | 13.92 | 100 | — |
| (DD) | | | | | | | |
| Y397V | 2.81 | 4.95 | 57 | 0.12 | 0.23 | 50 | — |
| Catalytic | | | | | | | |
| Triad | | | | | | | |
| S52A | 2.08 | 2.30 | 91 | 2.41 | 2.85 | 85 | — |
| S52A | 5.37 | 6.24 | 86 | 5.31 | 7.68 | 69 | — |
| Y397V | | | | | | | |
| S53A | 5.80 | 6.36 | 91 | 4.88 | 6.96 | 70 | — |
| S53A | 15.45 | 21.32 | 73 | 10.66 | 15.42 | 69 | — |
| Y397V | | | | | | | |
| S52A | 2.29 | 2.59 | 88 | 0.91 | 1.27 | 72 | 0.39 |
| S53A | | | | | | | |
| S52A | 3.01 | 4.54 | 66 | 0.11 | 0.30 | 36 | 0.36 |
| S53A | | | | | | | |
| Y397V | | | | | | | |
| H210V | 0.15 | 0.14 | 100 | 0.03 | 0.06 | 62 | 0.05 |
| H210V | 0.01 | 0.00 | 100 | 0.00 | 0.01 | 49 | 0.01 |
| Y297V | | | | | | | |
| H211V | 0.02 | 0.02 | 100 | 0.05 | 0.06 | 87 | 0.08 |
| H211V | 0.09 | 0.09 | 93 | 0.02 | 0.05 | 40 | 0.05 |
| Y397V | | | | | | | |
| D50A | 0.62 | 0.92 | 67 | 0.29 | 0.85 | 34 | — |
| D50A | 2.82 | 4.90 | 58 | 1.98 | 4.19 | 47 | — |
| Y397V | | | | | | | |
| E129A | 0.00 | 0.00 | — | 0.00 | 0.04 | 0 | — |
| E129A | 0.00 | 0.00 | — | 1.42 | 2.03 | 70 | — |
| Y397V | | | | | | | |
| E328V | 0.32 | 0.42 | 78 | 0.00 | 0.03 | 0 | — |
| E328V | 0.46 | 0.53 | 86 | 1.64 | 1.71 | 100 | — |
| Y397V | | | | | | | |
| E357A | 0.36 | 0.21 | 100 | 0.00 | 0.00 | — | — |
| E357A | 0.04 | 0.06 | 62 | 0.04 | 0.01 | 100 | — |
| Y397V | | | | | | | |

The results of the serine protease inhibitor experiments are shown in Table 16.

TABLE 16
HPLC data obtained in the experiments using the serine protease
inhibitors PMSF and AEBSF. Data is presented as a percentage reduction
in specific activity between the control reaction run in the absence of the
inhibitor and the reaction run in the presence of the inhibitor. Positive %
reduction indicates that the activity increased relative to activity in the absence of inhibitor.

| | | Mn | | | Mg | | |
|---|---|---|---|---|---|---|---|
| | | % Reduction | % Reduction | | % Reduction | % Reduction | |
| | | in ADP-based | in Glutamine- | | in ADP-based | in Glutamine- | |
| | | Specific | based Specific | | Specific | based Specific | |
| Enzyme | | Activity | Activity | % Conversion | Activity | Activity | % Conversion |
| WT (AD) | No | | | 101 | | | 116 |
| | Inhibitor | | | | | | |
| | PMSF | −15.63 | −49.20 | 61 | +48.13 | −46.33 | 42 |
| | AEBSF | −26.76 | −14.86 | 118 | −34.60 | −24.59 | 134 |
| WT (DD) | No | | | 92 | | | 76 |
| | Inhibitor | | | | | | |
| | PMSF | −3.53 | −17.69 | 78 | −6.07 | −16.55 | 67 |
| | AEBSF | −4.73 | −2.23 | 94 | −14.98 | −8.43 | 82 |
| Y397V | No | | | 91 | | | 108 |
| | Inhibitor | | | | | | |
| | PMSF | −17.58 | −44.47 | 61 | −4.88 | −31.39 | 78 |
| | AEBSF | −20.13 | −4.98 | 108 | −43.37 | −10.83 | 107 |
| S52A | No | | | 92 | | | 84 |
| | Inhibitor | | | | | | |
| | PMSF | −13.41 | −39.84 | 64 | −19.51 | −43.54 | 59 |
| | AEBSF | +30.66 | +15.27 | 81 | −31.77 | −10.61 | 109 |
| S52A | No | | | 80 | | | 100 |
| Y397V | Inhibitor | | | | | | |
| | PMSF | −15.50 | −29.12 | 67 | +73.28 | −77.41 | 49 |
| | AEBSF | −16.65 | −11.57 | 85 | −100.00 | +1.95 | — |
| S53A | No | | | 115 | | | 51 |
| | Inhibitor | | | | | | |
| | PMSF | +46.95 | −22.73 | 61 | −17.31 | +6.90 | 66 |
| | AEBSF | −15.43 | −15.19 | 115 | −39.50 | −1.70 | 83 |
| S53A | No | | | 82 | | | 105 |
| Y397V | Inhibitor | | | | | | |
| | PMSF | +39.67 | +6.07 | 63 | ND | ND | — |
| | AEBSF | +14.80 | +5.16 | 75 | −100.00 | −55.60 | — |
| S52A | No | | | 89 | | | 82 |
| S53A | Inhibitor | | | | | | |
| | PMSF | −13.56 | −16.92 | 85 | −23.74 | −23.24 | 82 |
| | AEBSF | −16.20 | −7.69 | 98 | −41.91 | −17.82 | 115 |
| S52A | No | | | 72 | | | — |
| S53A | Inhibitor | | | | | | |
| Y397V | | | | | | | |
| | PMSF | −4.83 | −18.20 | 62 | DI | DI | — |
| | AEBSF | −5.20 | −1.65 | 75 | DI | DI | — |

ND = Not Done
DI = Data Inaccurate; Extremely low activity levels obtained, making interpretation of the results inaccurate

The values are presented as a percentage in the reduction of activity between the result obtained in the absence of inhibitor and the result obtained in the presence of inhibitor. The percentage of conversion, reflecting the conversion efficiency of the enzymes is also shown. Positive % reduction values obtained indicate an increase in activity in the presence of the inhibitor, above the level obtained in the control. This was especially evident in the ATP hydrolysis reactions, indicating that the first step of the reaction occurs at an increased rate in the presence of the inhibitor possibly as a result of the hydrolysis of the γ-glutamyl phosphate as in all cases where the rate of hydrolysis of ATP increased there was a concomitant reduction in the conversion efficiency. A percentage of reduction of ±15% was interpreted as a reflection of the variability in the assay, and it was, therefore, assumed that the protease inhibitor was having little or no effect.

PMSF was found to inhibit the adenylylated WT enzyme, as well as the mutants Y397V, S52A, S52A Y397V, and S53A in both the Mn²⁺- and Mg²⁺-based assays. AEBSF appeared to cause inhibition in the adenylylated WT enzyme, as well as the mutants Y397V, S52A, S52A Y397V, S53A, S53A Y397V, S52A S53A and S52A S53A Y397V, with the level of inhibition being significantly higher in the ATP hydrolysis reaction. Where AEBSF did not cause a significant reduction in the glutamine-based specific activity, its presence in the active site did appear to increase the ADP-based specific activity. PMSF did not significantly inhibit the activity of S52A S53A or S52A S53A Y397V. This was to be expected, as both serines have been removed from the active site. The mechanism of action of AEBSF, therefore, appears to be different as it did inhibit both these mutant enzymes.

Both PMSF and AEBSF were found to bind to the active site while still allowing ATP hydrolysis to occur. This may be interpreted as competitive binding of the serine protease inhibitor to the glutamate site, while still allowing ATP hydrolysis to occur. It is important to note that in all cases, while inhibition was found to occur, there was a concomitant reduction in the conversion efficiency.

The amino acids that have been identified from the SDM data that make up the respective catalytic triads are S52, H211 and E327 for the adenylylated form of the enzyme and S53, H210 and D50 for the deadenylylated form of the enzyme. To analyze how the enzyme might switch between catalytic triads upon adenylylation, the crystal structure model 1f52.pdb (Gill H S and D Eisenberg (2001) Biochemistry 7: 1903-1912) obtained from the Brookhaven Protein Database was reduced to 4 subunits, designated A, B, G, and H, to analyze interactions of the subunits in the putative catalytic triad-based reaction mechanism. The adenylylated Tyr397 of subunit H is opposite a Trp57′ on subunit B, which is in the serine flexible loop. It is believed that on adenylylation, an interaction is created between the aromatic side chains of the adenylylated Tyr397 and the Trp57′, as well as an interaction between the adenyl residues between Tyr397 (Subunit H) and Tyr397 (Subunit A), which cause the enzyme to “switch” the catalytic triad.

It is proposed that on adenylylation of Tyr397 from residue A Trp57 on subunit G between the aromatic rings of the adenyl group of the ADP and the tryptophan residue allowing the serine containing flexible loop on subunit G to swivel bringing Ser52 into closer proximity to Glu327 on subunit H. If Tyr397 on subunit H is also adenylylated it also interacts with the adenyl ring on the Tyr 397 of subunit A bringing Glu327 on subunit H into closer proximity in the active site. This resulting change in configuration between the active sites residues that exist between subunits H and G as well as the adenylylation of the Tyr 397 on subunit A then allows for sufficient change in the internal structure of the active site allowing His211 to move into position.

Conclusions

Structural and molecular dynamics analyses of glutamine synthetase suggested that a possible mechanism by which the adenylylation/deadenylylation state of the enzyme affects the enzyme specificity for either MgATP or Mn₂ATP and NH₄⁺ or NH₃ is by causing a switch between two putative catalytic triads. If a catalytic triads play a role in the catalysis, the reaction should pass through a γ-glutamyl acyl enzyme intermediate.

Site-directed mutagenesis of a number of residues identified as playing a role in these catalytic triads led to the following observations. Both Ser52 and Ser53 were important for the catalytic activity of the enzyme. It was determined that Ser52 appeared to have adenylylated functionality and Ser53 deadenylylated functionality. It is possible, however, that as these serine residues lie adjacent to one another, both are capable of functioning in the adenylylated and deadenylylated forms of the enzyme. When both serine residues were removed, resulting in decreased glutamine synthetase activity, the importance of these residues in the active site was reinforced. Evaluating these serine mutant enzymes in the presence of the serine protease inhibitors, AEBSF and PMSF, led to the conclusion that Ser52 and Ser53, did indeed appear to form part of a catalytic triad, as the activity of the enzymes were inhibited in the presence of the inhibitors. The double serine mutant was not significantly inhibited by either PMSF or AEBSF. Both AEBSF and PMSF appeared to cause an increase in the rate of hydrolysis of the ATP with a concomitant reduction in the formation of glutamine and the conversion efficiency. This may be interpreted as competitive binding of the serine protease inhibitor to the glutamate site, while still allowing ATP hydrolysis to occur. It is important to note that in all cases, while inhibition was found to occur, there was a concomitant reduction in the conversion efficiency. His210 and His211 were found to be equally important to the functionality of the enzyme, and the results, in consultation with the model, led to the conclusion that His210 had deadenylylated activity and His211 had adenylylated activity. All the potential acid residues, when removed, had an effect on activity. Again, consultation of the model led to the conclusion that the two acid residues that filled the function of the acid residue in each catalytic triad, were Glu129 for the adenylylated form of the enzyme, and Glu357 for the deadenylylated form of the enzyme.

The two putative catalytic triads are found at the interface between two subunits (designated A and B) of glutamine synthetase and are believed to be comprised of the following residues (where the E. coli residue number is given)
Adenylylated GS:
   Ser52 (Subunit B)
   His211 (Subunit A)
   Glu129 (Subunit A)
Deadenylylated GS:
   Ser53 (Subunit B)
   His 210 (Subunit A)
   Glu357 (Subunit A).

The deadenylylated GS is produced under conditions of nitrogen limitation and at low NH₃ concentrations; the enzyme is deadenylylated, switching to the catalytic triad containing residue Ser53 (Subunit B); at high NH₃ concentrations, the enzyme is adenylylated, switching to the catalytic triad containing the residue Ser52. The catalytic site using Ser52′ must deprotonate the NH₄⁺ to create the ammonia nucleophile. It has been proposed that the deprotonation of the NH₄⁺ may occur via Asp50 (Liaw S-H, I Kuo and D Eisenberg (1995) Protein Science 4: 2358-2365). This deprotonation of the NH₄⁺ could, however, also occur via a carbonium ion once the acyl enzyme intermediate has been formed. The two proposed reaction mechanisms, whereby the nucleophilic attack by ammonia of a γ-glutamyl-acyl enzyme intermediate is facilitated by a catalytic triad mechanism, are discussed below. An acyl enzyme intermediate has been postulated before in work carried out on glutamine synthetase isolated from sheep brain, where ¹⁴C-glutamate binding to glutamine synthetase in the absence of NH₃ was demonstrated (Krishnaswamy P R, V Pamiljans and A Meister (1962) Journal of Biological Chemistry 237: 2932-2940).

As indicated above, the reaction kinetics of the nucleophilic attack by ammonia show two sets of kinetic constants. One proposed mechanism for the catalytic triad reaction is that one of the γ-oxygens of Ser52′ or Ser53′ forms a covalent bond to the glutamate, via nucleophilic attack on the carbonyl carbon of the ester bond of the carboxylic-phosphoric acid anhydride of the glutamyl phosphate formed in the first step of the reaction.

His210 and His211 act as general base catalysts by removing the proton from the respective Ser O^{γ}. This Ns-bound proton forms hydrogen bonds to the Ser O^{γ} and to the substrate phosphate oxygen. The resulting tetrahedral oxyanion intermediate is possibly stabilised by Arg339. In the next step, the N^{ε}-proton from His210 and His211 is transferred to the bridging oxygen substrate, releasing the acid phosphate, with the concomitant formation of an acyl enzyme intermediate. It is after the formation of the first acyl enzyme intermediate at either Ser52′ or Ser53′, that there appears to be a fundamental difference in the reaction mechanisms of the nucleophilic attack by ammonia and the subsequent deacylation of the enzyme and the formation of glutamine. It is believed that the “choice” of the serine is mediated by the adenylylation state of the active site and the use of either Mg²⁺ or Mn²⁺ in the transfer of phosphate from ATP to glutamate. This transfer of phosphate to the glutamate may occur via phosphoryl transfer mediated by His269, which has been identified as a ligand of the n2 metal ion (Abell, L M, J Schineller, P J Keck and J Villafranca (1995) Biochemistry 34: 16695-16702). The role of His269 in the phosphoryl transfer has also been demonstrated in a novel phosphoryl transferase reaction via His 269 (Kenyon, C. P., et al. (2006), PCT application entitled “MODULATION OF PHOSPHORYL TRANSFERASE ACTIVITY,” Attorney Docket Number 19690-004WO1, filed simultaneously herewith).

Another factor that may play a role in the switching mechanism is that Mn₂ATP has a different structure from MgATP, as the ATP folds around the Mn ions in a binary complex with the closest approach of the adenine ring being made by N₇. The Mn₂ATP and the MgATP may cause different oxygens of the Glu δ carbonyl group to be phosphorylated. In aqueous solution these would normally be equivalent. However, within the active site, as the acyl intermediate, the Glu O^{ε1} and Glu O^{ε2} are asymmetric in nature, maintaining their stereochemistry. This asymmetry may be important in deciding the nature of nucleophilic attack. It is therefore conceivable that either S_{N}1 or S_{N}2 nucleophilic attack may occur as a result of the orientation created by the phosphorylation of either Glu O^{ε1} or Glu O^{ε2}.

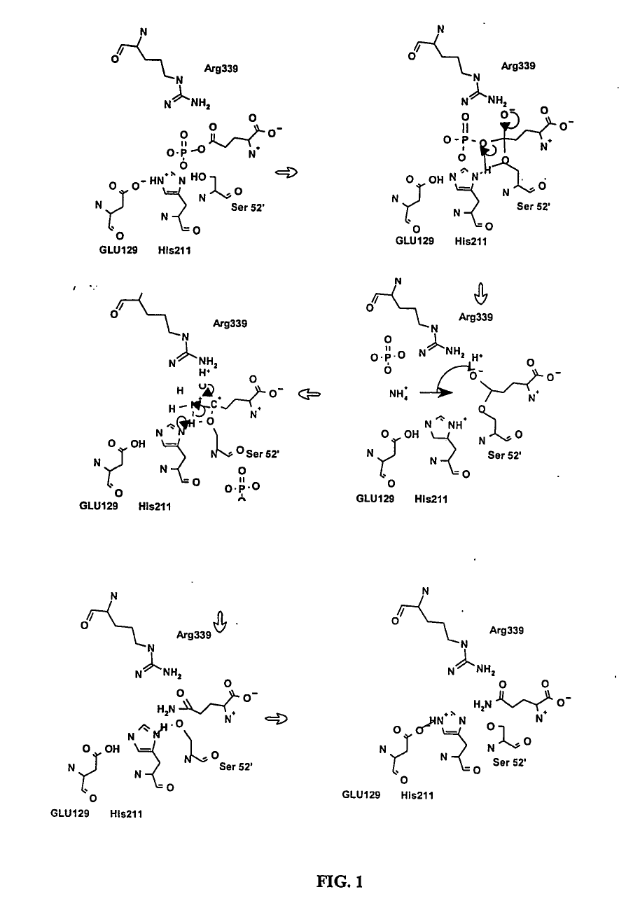

Thus, a possible model mechanism for the adenylylated form of the enzyme that is formed in the cells grown under conditions of nitrogen excess and carbon limitation, using the Ser52′/His211/Glu129 catalytic triad is outlined in FIG. 1. The γ-carboxyl group on the glutamate acyl intermediate may play a role in the deprotonation of the NH₄⁺. The first tetrahedral transition state occurs during the dephosphorylation of the γ-glutamyl phosphate. This proton, in activating this zwitter-ionic form of the carboxyl group, increases its susceptibility to nucleophilic attack. The overall activity of the adenylylated enzyme, in the presence of Mn₂ATP, was found to be generally lower than the deadenylylated enzyme. The high activation energy in the adenylylated enzyme, however, may be linked to the enzyme having to deprotonate the NH₄⁺ to NH₃+H⁺ at the high NH₄⁺ concentrations, to create the nucleophile.

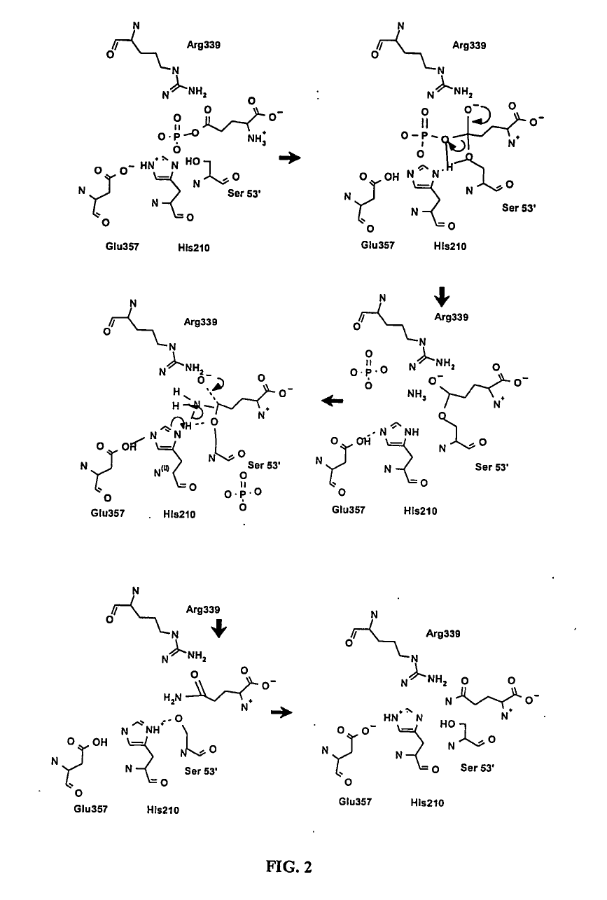

A possible model mechanism for the deadenylylated form of the enzyme that is formed in the cells grown under conditions of nitrogen limitation and carbon excess, using the Ser53′/His210/E357 catalytic triad is outlined in FIG. 2. After the formation of the first tetrahedral transition state, and after the N^{ε} proton of His210 has been transferred to the substrate, thereby releasing the acid phosphate, the acyl enzyme intermediate is formed. This acyl enzyme complex may be stabilised by the Arg339 in an oxyanion hole. The nucleophilic attack by the ammonia then occurs. As the deadenylylated form of the enzyme is produced by the cells under nitrogen limited conditions, it is believed that the NH₄⁺ is fully dissociated to NH₃+H⁺, with NH₃ being brought into the active site and not NH₄⁺.

It has been shown that an aqueous solution of (NH₄)₂SO₄ dissociates into 2NH₄⁺ and SO₄⁻. However, in dilute solutions, as the concentration tends towards infinite dilution, it is believed that the 2NH₄⁺ further dissociates to 2NH₃ and 2H⁺. Therefore, a possible third mechanism involved in facilitating the nucleophilic attack of the acyl enzyme by the ammonia may occur. This is a “gas phase” or “solvent free” reaction. The rates of bimolecular nucleophilic substitution reactions involving anions and polar molecules vary over 20 orders of magnitude in changing from the gas phase to polar media (solvent) (Olmstead, W. N. and J. I. Brauman (1977) J. Am. Chem. Soc. 99: 4219-4228; Pellerite, M. J. and J. I. Brauman (1980) J. Am. Chem. Soc. 102: 5993-5999; Shaik, S. S, and A. Pross (1982) J. Am. Chem. Soc. 104: 2708-2719; Chandrasekhar, J., Smith, S. F. and W. L. Jorgensen (1984) J. Am. Chem. Soc. 106: 3049-3050; Chandrasekhar, J., Smith, S. F. and W. L. Jorgensen (1985) J. Am. Chem. Soc. 107: 154-163; Chandrasekhar, J. and W. L. Jorgensen (1985) J. Am. Chem. Soc. 107: 2974-2975). This is believed to be due to the increase in the activation energy by hydration as a result of the reduction in the strength of the hydrogen bonds. The ion-dipole attraction is nullified by the energy required to desolvate the ion (nucleophile). The transition state, which has a dispersed charge distribution as a result of the hydration forms weaker hydrogen bonds to the solvent. This results in a unimodal energy profile with a large, narrow central energy barrier that is significantly greater than in the gas phase. It is therefore postulated, that at very low ammonia concentrations the active site is “closed”, thereby reducing or eliminating the solvent accessible area in the active site, allowing a gas phase type reaction to occur. This would possibly occur when the enzyme is deadenylylated and functioning in the Mg²⁺ form. It would also account for the very low Kₘ which the enzyme has for ammonia at low substrate concentrations. The adverse effect of H₂O was evident in the enzyme reactions of the mutant enzymes Y397V, S52A S53A Y397V, H210V Y397V and H211V Y397V as the conversion efficiency of ATP hydrolysed to glutamine formed was as low as 36%.

Overall, the data show that the reaction mechanism employed by glutamine synthetase in the formation of glutamine from the γ-glutamyl phosphate synthesized in the first step of the reaction occurs via two catalytic triads similar to those employed by serine proteases. These catalytic triads include Ser52′, His211 and Glu129 for the adenylylated form of the E. Coli enzyme, and Ser53′, His210 and Glu357 for the deadenylylated form of the E. coli enzyme.

A number of embodiments of the invention have been described. Nevertheless, it will be understood that various modifications may be made without departing from the spirit and scope of the invention. Accordingly, other embodiments are within the scope of the following claims.

What is claimed is:

1. A computer-assisted method of generating a test inhibitor of the glutamine formation active site activity of a glutamine synthetase polypeptide, the method using a programmed computer comprising a processor and an input device, the method comprising:
(a) inputting on the input device data comprising a structure of a glutamine formation active site;
(b) docking into the glutamine formation active site a test inhibitor molecule using the processor; and
(c) determining, based on the docking, whether the test inhibitor molecule would inhibit the glutamine formation active site activity.

2. The method of claim 1, further comprising docking into the active site a γ-glutamyl phosphate moiety.

3. The method of claim 1, further comprising producing a test inhibitor determined by step (c) to inhibit the glutamine formation active site activity and evaluating the inhibitory activity of the test inhibitor on a glutamine synthetase polypeptide in vitro.

4. The method of claim 3, wherein said in vitro evaluation comprises use of an assay capable of measuring ATP hydrolysis, ADP formation, AMP formation, glutamate utilization, or glutamine formation.

5. The method of claim 3, further comprising evaluating the differential inhibitory activity of the test inhibitor on an adenylylated glutamine synthetase polypeptide relative to a deadenylylated glutamine synthetase polypeptide in vitro.

6. The method of claim 1, further comprising producing the test inhibitor and evaluating the inhibitory activity of the test inhibitor on the growth of a bacterium comprising a GSI-α or a GSI-β glutamine synthetase gene.

7. The method of claim 6, wherein said GSI-β bacterium is selected from the group consisting of Corynebacterium diphtheriae, Neisseria gonorrhoeae, Escherichia coli, Salmonella typhinurium, Salmonella typhi, Klebsiella pneumoniae, Serratia marcescens, Proteus vulgaris, Shigella dysenteriae, Vibrio cholerae, Pseudomonas aeruginosa, Alcaligenes faecalis, Helicobacter pylori, Haemophilus influenzae, Bordetella pertussis, Bordella bronchiseptia, Neisseria meningitides, Brucella melitensis, Mycobacterium tuberculosis, Mycobacterium leprae, Treponema pallidum, Leptospira interrogans, Acetinomyces israelii, Nocardia esteroides, Thiobacillus ferrooxidans, Azospirillum brazilensis, Anabaena sp., Fremyella diplosiphon, and Streptomyces coelicolor, and wherein said GSI-α bacterium is selected from the group consisting of Bacillus cereus, Bacillus subtilis, Bacillus anthracis, Streptococcus pneumoniae, Streptococcus pyogenes, Staphylococcus aereus, Clostridium botulinum, Clostridium tetani and Clostridium perfringens.

8. The method of claim 7, wherein said GSI-β bacterium is Mycobacterium tuberculosis.

9. The method of claim 6, further comprising evaluating the inhibitory activity of the test inhibitor on the growth of a eukaryotic cell.

10. The method of claim 9, wherein said eukaryotic cell is a mammalian cell.

11. A method of generating a compound that inhibits the glutamine formation active site activity of a glutamine synthetase polypeptide, the method comprising:
(a) providing a three-dimensional structure of a glutamine formation active site of a glutamine synthetase polypeptide; and
(b) designing, based on the three-dimensional structure, a test compound capable of inhibiting the interaction between the glutamine formation active site and a γ-glutamyl phosphate intermediate.

12. The method of claim 11, wherein said test compound is capable of inhibiting the interaction between an adenylylated catalytic triad site of the glutamine formation active site and a γ-glutamyl phosphate intermediate, or of inhibiting the interaction between an deadenylylated catalytic triad site of the glutamine formation active site and a γ-glutamyl phosphate intermediate.

13. The method of claim 11, wherein said test compound is capable of inhibiting the formation of an acyl-enzyme intermediate in the adenylylated catalytic triad site of the glutamine formation active site, or of inhibiting the formation of the acyl-enzyme intermediate in the deadenylylated catalytic triad site of the glutamine formation active site.

14. The method of claim 12 or 13, further comprising producing the test compound of step (b) and evaluating the inhibitory activity of the test compound on a glutamine synthetase polypeptide in vitro.

15. The method of claim 12 or 13, further comprising producing the test compound of step (b) and evaluating the inhibitory activity of the test compound on the growth of a bacterium comprising a GSI-α or a GSI-β glutamine synthetase gene.

16. The method of claim 15, wherein said bacterium comprising a GSI-β gene is selected from the group consisting of Corynebacterium diphtheriae, Neisseria gonorrhoeae, Escherichia coli, Salmonella typhinurium, Salmonella typhi, Klebsiella pneumoniae, Serratia marcescens, Proteus vulgaris, Shigella dysenteriae, Vibrio cholerae, Pseudomonas aeruginosa, Alcaligenes faecalis, Helicobacter pylori, Haemophilus influenzae, Bordetella pertussis, Bordella bronchiseptia, Neisseria meningitides, Brucella melitensis, Mycobacterium tuberculosis, Mycobacterium leprae, Treponema pallidum, Leptospira interrogans, Acetinomyces israelii, Nocardia esteroides, Thiobacillus ferrooxidans, Azospirillum brazilensis, Anabaena sp., Fremyella diplosiphon, and Streptomyces coelicolor, and wherein said bacterium comprising a GSI-α gene is selected from the group consisting of Bacillus cereus, Bacillus subtilis, Bacillus anthracis, Streptococcus pneumoniae, Streptococcus pyogenes, Staphylococcus aereus, Clostridium botulinum, Clostridium tetani and Clostridium perfringens.

17. The method of claim 15, further comprising evaluating the inhibitory activity of the test compound on the growth of a eukaryotic cell.

18. The method of claim 17, wherein said eukaryotic cell is a mammalian cell.

19. A method of generating a test compound that inhibits a catalytic triad site activity of a glutamine formation active site of a glutamine synthetase polypeptide, the method comprising:
(a) providing a three-dimensional structure comprising a catalytic triad site of a glutamine formation active site; and
(b) designing, based on the three-dimensional structure, a test compound capable of forming an acyl-enzyme intermediate with a residue of the catalytic triad site.

20. The method of claim 19, wherein said test compound is capable of forming an acyl-enzyme intermediate with a residue in said structure corresponding to Ser52 or Ser53 of the glutamine synthetase polypeptide of E. coli.

21. A method of screening a test protease inhibitor compound in vitro to determine whether or not it inhibits the glutamine formation active site activity of a glutamine synthetase polypeptide, the method comprising:
(a) contacting a glutamine synthetase polypeptide with a test protease inhibitor compound; and
(b) determining whether or not the glutamine formation active site activity of the glutamine synthetase polypeptide is reduced relative to the activity of a glutamine synthetase polypeptide that has not been contacted with the test serine protease inhibitor compound.

22. The method of claim 21, wherein said glutamine formation site activity is measured by using an assay capable of measuring ATP hydrolysis, ADP formation, AMP formation, glutamate utilization, or glutamine formation.

23. The method of claim 21, wherein said test protease inhibitor compound is a test serine protease inhibitor compound.

24. The method of claim 23, wherein in step (a), a library of test protease inhibitor compounds are contacted, individually, with the glutamine synthetase polypeptide.

25. The method of claim 24, wherein the library is a library of test serine protease inhibitor compounds.

26. An in vitro method for inhibiting the glutamine formation active site activity of a GS polypeptide, the method comprising contacting a GS polypeptide with a composition comprising a serine protease inhibitor.

27. The method of claim 26, wherein said serine protease inhibitor is a peptidyl-arginine aldehyde derivative, a pyrrolopyrrolidene derivative, a quinolinone derivative, or a 1-methyl benzimidazole derivative.

28. An in vitro method for inhibiting growth of a bacterium comprising a GSI-α or a GSI-β gene, the method comprising contacting the bacterium with a composition comprising a serine protease inhibitor.

29. A method for treating, preventing, or ameliorating one or more symptoms or disorders associated with a bacterial infection in a mammal, wherein the bacterial infection is from a bacterium comprising a GSI-α or a GSI-β gene, the method comprising administering to the mammal a composition comprising a serine protease inhibitor.

30. An in vivo method for inhibiting the glutamine formation site activity of a glutamine synthetase polypeptide, the method comprising administering a composition comprising a serine protease inhibitor to a mammal that is suspected of suffering or is suffering from a bacterial infection, wherein the bacterial infection is from a bacterium comprising a GSI-α or GSI-β gene.

31. The method of any of claims 28-30, wherein said bacterium comprising a GSI-β gene is selected from the group consisting of Corynebacterium diphtheriae, Neisseria gonorrhoeae, Escherichia coli, Salmonella typhinurium, Salmonella typhi, Klebsiella pneumoniae, Serratia marcescens, Proteus vulgaris, Shigella dysenteriae, Vibrio cholerae, Pseudomonas aeruginosa, Alcaligenes faecalis, Helicobacter pylori, Haemophilus influenzae, Bordetella pertussis, Bordella bronchiseptia, Neisseria meningitides, Brucella melitensis, Mycobacterium tuberculosis, Mycobacterium leprae, Treponema pallidum, Leptospira interrogans, Acetinomyces israelii, Nocardia esteroides, Thiobacillus ferrooxidans, Azospirillum brazilensis, Anabaena sp., Fremyella diplosiphon, and Streptomyces coelicolor.

32. The method of claim 29 or 30, wherein said mammal is a human.


SEQUENCE LISTING (27 sequences)

SEQ ID NO: 1 - DNA, 21 bp, Artificial sequence
  Primer was designed to the sequence of the E.coli glnA gene obtained from Genbank (Accession Number X05173)
gatatgcatccgtcaaatgcg

SEQ ID NO: 2 - DNA, 22 bp, Artificial sequence
  Primer was designed to the sequence of the E.coli glnA gene obtained from Genbank (Accession Number X05173)
gcgatgcataaagtttccacgg

SEQ ID NO: 3 - DNA, 21 bp, Artificial sequence
  Sequence-specific primers used for sequencing of the clonedE.coli glnA gene
gctgaacacgtactgacgatg

SEQ ID NO: 4 - DNA, 23 bp, Artificial sequence
  Sequence-specific primer used for sequencing of the cloned E.coliglnA gene
gtgggaaccggagatagatgatc

SEQ ID NO: 5 - DNA, 22 bp, Artificial sequence
  Sequence-specific primer used for sequencing of the cloned E.coli glnA gene
cgatgttcggtgataacggctc

SEQ ID NO: 6 - DNA, 24 bp, Artificial sequence
  Sequence-specific primer used for sequencing of the cloned E.coliglnA gene
cgtacttcgatacgacgtgctttc

SEQ ID NO: 7 - DNA, 30 bp, Artificial sequence
  E. coli glnA Y397V mutagenesis primer
tggacaaaaacctggtcgacctgccgccag

SEQ ID NO: 8 - DNA, 31 bp, Artificial sequence
  E. coli glnA S52A mutagenesis primer
aaatgtttgacggcgcatcgattggcggctg

SEQ ID NO: 9 - DNA, 28 bp, Artificial sequence
  E. coli glnA S53A mutagenesis primer
gtttgacggatccgctattggcggctgg

SEQ ID NO: 10 - DNA, 27 bp, Artificial sequence
  E. coli glnA S52A S53A mutagenesis primer
gtttgacggcgcagcgatcggcggctg

SEQ ID NO: 11 - DNA, 32 bp, Artificial sequence
  E. coli glnA H210V mutagenesis primer
ggttgaagcccatgtccacgaagtcgcgactg

SEQ ID NO: 12 - DNA, 32 bp, Artificial sequence
  E. coli glnA H211V mutagenesis primer
gaagcccatcacgtcgaagtagcgacagctgg

SEQ ID NO: 13 - DNA, 31 bp, Artificial sequence
  E. coli E327A mutagenesis primer
ggtcccgggctatgtagcatcgataatgctg

SEQ ID NO: 14 - DNA, 21 bp, Artificial sequence
  Sequence-specific primer used for sequencing of the cloned E.coliglnA gene
gctgaacacgtactgacgatg

SEQ ID NO: 15 - DNA, 20 bp, Artificial sequence
  Sequence-specific primer used for sequencing of the cloned E.coliglnA gene
gtctgtctgagcaggcgctg

SEQ ID NO: 16 - DNA, 20 bp, Artificial sequence
  Sequence-specific primer used for sequencing of the cloned E.coli glnA gene
gctatcgacgatatcgaagg

SEQ ID NO: 17 - DNA, 23 bp, Artificial sequence
  Sequence-specific primer used for sequencing of the cloned E.coli glnA gene
gtgggaaccggagatagatgatc

SEQ ID NO: 18 - DNA, 20 bp, Artificial sequence
  Sequence-specific primer used for sequencing of the cloned E.coli glnA gene
gcataaagtttccacggcaa

SEQ ID NO: 19 - DNA, 22 bp, Artificial sequence
  Sequence-specific primer used for sequencing of the cloned E.coli glnA gene
gttcttgataccatcaagaccg

SEQ ID NO: 20 - DNA, 42 bp, Artificial sequence
  E. coli glnA D50A mutagenesis primer
gaagaaggcaaaatgtttgcaggctcatcgattggcggctgg

SEQ ID NO: 21 - DNA, 42 bp, Artificial sequence
  E. coli glnA D50A complementaty mutagenesis primer
ccagccgccaatcgatgagcctgcaaacattttgccttcttc

SEQ ID NO: 22 - DNA, 36 bp, Artificial sequence
  E. coli glnA E129A mutagenesis primer
ccgtactgttcgggcccgctcctgaattcttcctgt

SEQ ID NO: 23 - DNA, 35 bp, Artificial sequence
  E. coli glnA E128A complemetary mutagenesis primer
acaggaagaattcaggagcgggcccgacagtacgg

SEQ ID NO: 24 - DNA, 36 bp, Artificial sequence
  E. coli glnA E327A mutagenesis primer
gtctggtcccgggctatgtagcatcgataatgctgg

SEQ ID NO: 25 - DNA, 40 bp, Artificial sequence
  E. coli glnA E327A complemetary mutagenesis primer
cggttagctgcaggatccgggaaacgtaccgcgatacgac

SEQ ID NO: 26 - DNA, 40 bp, Artificial sequence
  E. coli glnA E357A mutagenesis primer
gtcgtatcgcggtacgtttcccggatcctgcagctaaccg

SEQ ID NO: 27 - DNA, 40 bp, Artificial sequence; identical to SEQ ID NO: 25
  E. coli glnA E357A complemetary mutagenesis primer